(19) United States
(12) Patent Application Publication
(10) Pub. No.: US 2024/0382307 A1
(43) Pub. Date: Nov. 21, 2024

(54) PROSTHETIC VALVE HAVING LAMINATED SKIRT ASSEMBLY

(71) Applicant: EDWARDS LIFESCIENCES CORPORATION, Irvine, CA (US)

(72) Inventors: Tamir S. Levi, Zikhron Yaakov (IL); Michael Bukin, Pardes Hanna (IL); Nikolai Gurovich, Hadera (IL); Ziv Yohanan, Kfar Hahoresh (IL)

(21) Appl. No.: 18/788,468

(22) Filed: Jul. 30, 2024

Related U.S. Application Data
(63) Continuation of application No. PCT/US2023/012269, filed on Feb. 3, 2023.
(60) Provisional application No. 63/267,535, filed on Feb. 3, 2022.

(51) Int. Cl. A61F 2/24

(52) U.S. Cl. CPC A61F 2/2418; A61F 2210/0076; A61F 2240/001

(57) ABSTRACT

An implantable prosthetic valve including a plurality of leaflets and an annular frame that is radially expandable from a radially compressed state to a radially expanded state. The prosthetic valve also includes a skirt assembly comprising a reinforcing layer sandwiched between a first encapsulating layer and a second encapsulating layer, which are made of a thin elastomeric material. The reinforcing layer comprises a first set of yarns and a second set of yarns interwoven in a leno weave pattern, wherein the first and second sets of yarns are non-perpendicular and non-parallel to a longitudinal axis of the frame when the valve is in the radially expanded state. The first encapsulating layer and the second encapsulating layer can be fused or bonded along specified regions and form one or more pockets that enable free movement of sections of the reinforcing layer disposed therein.

### CROSS-REFERENCE TO RELATED APPLICATIONS

This application is a continuation of PCT Application No. PCT/US2023/012269, filed Feb. 3, 2023, which claims the benefit of U.S. Provisional Patent Application No. 63/267,535, filed Feb. 3, 2022, the entire contents of each of which are incorporated herein by reference.

### FIELD

The present disclosure relates to prosthetic heart valves, in particular, encapsulated skirts (for example, inner and outer skirts) that have an overall reduced thickness and increased stretchability that enable transition of the skirt to an axially elongated position when the prosthetic valves are in a crimped, radially compressed state.

### BACKGROUND

The human heart can suffer from various valvular diseases, which may result in significant malfunctioning of the heart and ultimately require repair of the native valve or replacement of the native valve with an artificial (prosthetic) valve. There are a number of known repair devices (for example, stents) and artificial valves, as well as a number of known methods of implanting these devices and artificial valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped configuration or state on the end of a delivery device and advanced through the patient's vasculature until the prosthetic valve reaches the implantation site in the heart. The prosthetic valve is then radially expanded to its functional size, for example, by inflating a balloon on which the prosthetic valve is mounted, actuating a mechanical actuator that applies an expansion force to the prosthetic valve, or by deploying the prosthetic valve from a sheath of the delivery device so that the prosthetic valve can self-expand to its functional size.

Such expandable, transcatheter prosthetic heart valves can include an annular metal frame, a valvular structure having a plurality of leaflets supported within the frame, an inner skirt coupled to an interior of the metal frame, and an outer skirt coupled to an exterior of the metal frame. The inner skirt can serve several functions. For example, the inner skirt can function as a seal member to prevent (or decrease) perivalvular leakage, to anchor the leaflets to the frame, and to protect the leaflets against damage caused by contact with the frame during crimping and during working cycles of the prosthetic valve. The outer skirt can cooperate with the inner skirt to further reduce or avoid perivalvular leakage after implantation of the prosthetic valve. The inner skirt may be comprised of a tough, tear resistant material such as polyethylene terephthalate (PET), although various other synthetic or natural materials can be used.

The inner and outer skirts are often constructed of a semi-porous material, for example, woven polyethylene terephthalate (PET). The porous nature of the skirt material is designed to encourage cellular ingrowth of the surrounding native tissue or from surrounding overgrown devices. This tissue ingrowth into the skirts may integrate the implanted heart valve within the native anatomy of the patient and further reduce paravalvular leakage (PVL). However, such ingrowth may propagate from the porous skirts onto the leaflets of the implanted prosthetic heart valve. Indeed, growths of tissue and pannus have been observed on leaflets in implanted prosthetic heart valves, which can act as substrates onto which thrombi may later deposit.

Further, the inner and outer skirts are frequently secured to the frame by suturing or stitching the fabric of the respective skirts to the frame. Suturing of the inner skirt to the frame can expose the leaflets to the sutures. During working cycles of the valve, repetitive contact between the leaflets and the exposed sutures as well as contact between the leaflets and the fabric material of the skirt can cause abrasion of the leaflets. In order to address such issues, the inner skirt and/or the outer skirt can be encapsulated. However, encapsulation of the skirts can have undesirable effects.

Accordingly, improvements to skirts for prosthetic valves are desirable.

### SUMMARY

Described herein are prosthetic heart valves, and methods for assembling prosthetic heart valves. The disclosed prosthetic heart valves and methods can, for example, enable an encapsulated outer skirt on a prosthetic valve to have a reduced thickness and volume when in an axially elongated state (corresponding to a crimped state for a prosthetic valve) to reduce an overall crimp profile of the prosthetic valve. In another example, the encapsulated outer skirt configurations disclosed herein can enable increased flexibility and/or stretchability of an encapsulated outer skirt during transitioning of a prosthetic valve between a crimped state and a radially expanded state. As such, the devices and methods disclosed herein can, among other things, overcome one or more of the deficiencies of typical prosthetic heart valves and their delivery and implantation.

A prosthetic heart valve can comprise a frame and a valve structure (for example, a plurality of leaflets) coupled to the frame. In addition to these components, a prosthetic heart valve can further comprise one or more of the components disclosed herein.

In some examples, a prosthetic heart valve can comprise a radially expandable and compressible frame.

In some examples, a prosthetic heart valve can comprise a skirt assembly.

In some examples, a skirt assembly for a prosthetic heart valve can comprise a reinforcing layer sandwiched between a first encapsulating layer and a second encapsulating layer, wherein the reinforcing layer comprises a textile comprising a first set of yarns and a second set of yarns interwoven with the first set of yarns, and wherein the skirt assembly has a first thickness at a plurality of relatively thinner areas between adjacent yarns and a second thickness at the yarns, wherein the first thickness is less than the second thickness.

In some examples, a reinforcing layer of a skirt assembly comprises a textile comprising a first set of yarns and a second set of yarns that are non-perpendicular and non-parallel to a longitudinal axis of a prosthetic heart valve when a frame of the prosthetic heart valve is in a radially expanded state.

In some examples, a reinforcing layer of a skirt assembly comprises a textile comprising a first set of yarns and a second set of yarns wherein spacing between adjacent yarns in the first set of yarns is in a range of about 0.5 mm to about 2.0 mm.

In some examples, yarns of a first set of yarns in a textile for a reinforcing layer of a skirt assembly have a thickness or diameter in a range of about 8 μm to about 16 μm.

In some examples, yarns of a second set of yarns in a textile for a reinforcing layer of a skirt assembly have a thickness or diameter in a range of about 8 μm to about 16 μm.

In some examples, a reinforcing layer of a skirt assembly comprises a textile having a weave density of less than 150 ppi.

In some examples, a first encapsulating layer of a skirt assembly comprises an elastomer and has a thickness in a range of about 5 μm to about 15 μm.

In some examples, a second encapsulating layer of a skirt assembly comprises an elastomer and has a thickness in a range of about 5 μm to about 15 μm.

In some examples, a skirt assembly has a first thickness at a plurality of relatively thinner areas between adjacent yarns and a second thickness at the yarns, wherein the first thickness is less than the second thickness, and the first thickness is in a range of about 10 μm to about 30 μm.

In some examples, a skirt assembly for a prosthetic heart valve can comprise a reinforcing layer sandwiched between a first encapsulating layer and a second encapsulating layer, wherein the first and second encapsulating layers are bonded together along one or more bonded regions, and one or more pockets are formed in areas between the one or more bonded regions, thereby enabling sections of the reinforcing layer within the one or more pockets to be unattached to the first and second encapsulating layers.

In some examples, the reinforcing layer can move relative to the first and second encapsulating layers within the one or more pockets.

In some examples, the first and second encapsulating layers are fused to each other at the one or more bonded regions.

In some examples, the one or more bonded regions include a first bonded region along a first circumferential edge of the skirt assembly and a second bonded region along a second circumferential edge of the skirt assembly.

In some examples, the one or more bonded regions are shaped to correspond to cusp edge portions of leaflets sutured to the skirt assembly along the bonded regions.

In some examples, a prosthetic heart valve comprises one or more of the components recited in Examples 1-94.

In some examples, a prosthetic valve comprises an annular frame that is radially expandable from a radially compressed state to a radially expanded state, the annular frame having an inflow end, an outflow end, and a longitudinal axis extending from the inflow end to the outflow end; a plurality of leaflets configured to regulate a flow of blood from the inflow end to the outflow end of the annular frame; and a skirt assembly comprising a reinforcing layer sandwiched between a first encapsulating layer and a second encapsulating layer, wherein the reinforcing layer comprises a textile comprising a first set of yarns and a second set of yarns interwoven with the first set of yarns. In some examples, the skirt assembly has a first thickness at a plurality of relatively thinner areas between adjacent yarns and a second thickness at the yarns, wherein the first thickness is less than the second thickness.

In some examples, a prosthetic valve comprises an annular frame that is radially expandable from a radially compressed state to a radially expanded state, the annular frame having an inflow end, an outflow end, and a longitudinal axis extending from the inflow end to the outflow end; a plurality of leaflets configured to regulate a flow of blood from the inflow end to the outflow end of the annular frame; and a skirt assembly comprising a reinforcing layer sandwiched between a first encapsulating layer and a second encapsulating layer, wherein the reinforcing layer comprises a textile comprising a first set of yarns and a second set of yarns interwoven in a leno weave pattern with the first set of yarns, wherein the first set of yarns and the second set of yarns are each non-perpendicular and non-parallel to the longitudinal axis when the annular frame is in the radially expanded state. In examples, each of the first encapsulating layer and the second encapsulating layer is made of an elastomer and has a thickness in a range of about 5 μm to about 15 μm.

In some examples, a prosthetic valve comprises an annular frame that is configured to be radially expanded from a radially compressed state to a radially expanded state, the annular frame having an inflow end and an outflow end; a plurality of leaflets configured to regulate a flow of blood therethrough; and a skirt assembly comprising a reinforcing layer sandwiched between a first encapsulating layer and a second encapsulating layer. In examples, the skirt assembly is configured such that the first encapsulating layer and the second encapsulating layer are fused along one or more bonded regions, and one or more pockets are formed in areas between the one or more bonded regions, thereby enabling sections of the reinforcing layer within the one or more pockets to be unattached to the first encapsulating layer and the second encapsulating layer.

In some examples, a skirt assembly for a prosthetic valve is configured to be attached to a surface of an annular frame and the prosthetic valve and to be moveable between a relaxed state and an axially elongated state. The skirt assembly comprises a first encapsulating layer and a second encapsulating layer, each of the first encapsulating layer and the second encapsulating layer comprising an elastomeric material; and a reinforcing layer disposed between the first encapsulating layer and the second encapsulating layer, wherein the reinforcing layer comprises a textile comprising a first set of yarns and a second set of yarns interwoven with the first set of yarns, wherein the first set of yarns and the second set of yarns are each non-perpendicular and non-parallel to a top edge and a bottom edge of the skirt assembly when the skirt assembly is in the relaxed state. In examples, the skirt assembly is configured such that the first encapsulating layer and the second encapsulating layer are fused along one or more bonded regions, thereby forming one more pockets between the encapsulating first layer and the second encapsulating layer, wherein the first and second encapsulating layers are not fused to each other within the pockets.

In some examples, a method of assembling a prosthetic valve comprises mounting a plurality of leaflets to an annular frame that is radially expandable from a radially compressed state to a radially expanded state, wherein the frame has an inflow end, an outflow end, and a longitudinal axis extending from the inflow end to the outflow end, wherein the plurality of leaflets are configured to regulate a flow of blood from the inflow end to the outflow end of the frame; forming a skirt assembly comprising a woven reinforcing layer sandwiched between a first encapsulation layer and a second encapsulation layer, the forming comprising fusing the first encapsulation layer and the second encapsulation layer along one or more fused regions, the one or more fused regions comprising a first region along a top edge of the skirt assembly and a second region at along a bottom edge of the skirt assembly; and mounting the skirt assembly to the annular frame.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 shows a side elevation view of an exemplary implantable prosthetic valve.

FIG. 2 shows a top perspective view of the prosthetic valve of FIG. 1.

FIG. 3 shows a top perspective view of an exemplary frame of the prosthetic valve of FIG. 1.

FIG. 4 shows a flattened view of the frame shown in FIG. 3.

FIG. 5 shows is a cross-sectional view of the prosthetic valve of FIG. 1 taken along line 5-5 of FIG. 1.

FIG. 6 shows the formation of a first encapsulating layer on a mandrel according to an exemplary process of making the inner skirt of the prosthetic valve of FIG. 1.

FIG. 7 shows the placement of a reinforcing layer over the first encapsulating layer shown in FIG. 6 according to the exemplary process of making the inner skirt.

FIG. 8 shows the placement of a plurality of masks over the reinforcing layer shown in FIG. 7 according to the exemplary process of making the inner skirt.

FIG. 9 shows the formation of a second encapsulating layer over the reinforcing layer having masks shown in FIG. 8 according to the exemplary process of making the inner skirt.

FIG. 10 shows the removal of the masks after forming the second encapsulating layer shown in FIG. 9 according to the exemplary process of making the inner skirt.

FIG. 11 is a cross-sectional view of a portion of an inner skirt having a window on one side of the inner skirt exposing the underlying woven fabric of the reinforcing layer.

FIG. 12 is a cross-sectional view showing threading of a suture through the woven fabric of the reinforcing layer at the window shown in FIG. 11.

FIG. 13 is a cross-sectional view showing suturing the inner skirt shown in FIG. 11 to an adjacent strut of a prosthetic valve's frame.

FIG. 14 is a perspective view of a prosthetic valve, according to another example.

FIG. 15 is a cross-sectional view of the prosthetic valve of FIG. 14 taken along line 15-15 of FIG. 14.

FIGS. 16A and 16B show portions of two interwoven yarns of a skirt when the skirt is in a relaxed state (corresponding to a radially expanded state of a prosthetic valve) and an axially stretched state (corresponding to a radially compressed state of the prosthetic valve), respectively.

FIG. 17 shows a section of a braided layer for the outer skirt of the prosthetic valve of FIGS. 14-15, according to one example.

FIG. 18 shows a section of a plain weave layer for the outer skirt of the prosthetic valve of FIGS. 14-15, according to another example.

FIGS. 19A-19J illustrate sections of various leno weave layers and associated leno weaving techniques for the outer skirt of the prosthetic valve of FIGS. 14-15, according to other examples.

FIG. 20A is a schematic illustration of a section of an encapsulated skirt, according to another example.

FIG. 20B is a cross-sectional view of a section of the exemplary encapsulated skirt shown in FIG. 20A.

FIG. 21 is a plan view schematic illustration of a section of an encapsulated skirt having pockets formed therein, according to another example.

FIG. 22 is a schematic illustration of an assembly for forming a skirt assembly for a prosthetic valve, according to one example.

FIG. 23 is a plan view of a pressing plate that can be used in the assembly of FIG. 22 for forming a skirt assembly.

### DETAILED DESCRIPTION

As discussed above, encapsulation of the inner and/or outer skirts in prosthetic valves may have undesirable effects. For example, encapsulation may cause the skirts to become overly rigid, which may thereby inhibit transition between a relaxed state and an axially elongated state of the skirt during transition of the prosthetic valve between radially expanded state and the crimped state. In other examples, the encapsulation of the inner and/or outer skirt can increase volume of the skirt material, and thereby undesirably increases a profile of a prosthetic valve in a crimped state, which may for example, make transcatheter delivery and manipulation of the prosthetic valve more difficult.

The encapsulated skirts disclosed herein address the foregoing issues. For example, the skirt configurations disclosed herein can enable an encapsulated outer skirt to have a reduced thickness and volume when in an axially elongated state (corresponding to a crimped state for a prosthetic valve). In another example, the skirt configurations disclosed herein can enable increased flexibility and/or stretchability of an encapsulated outer skirt.

### General Considerations

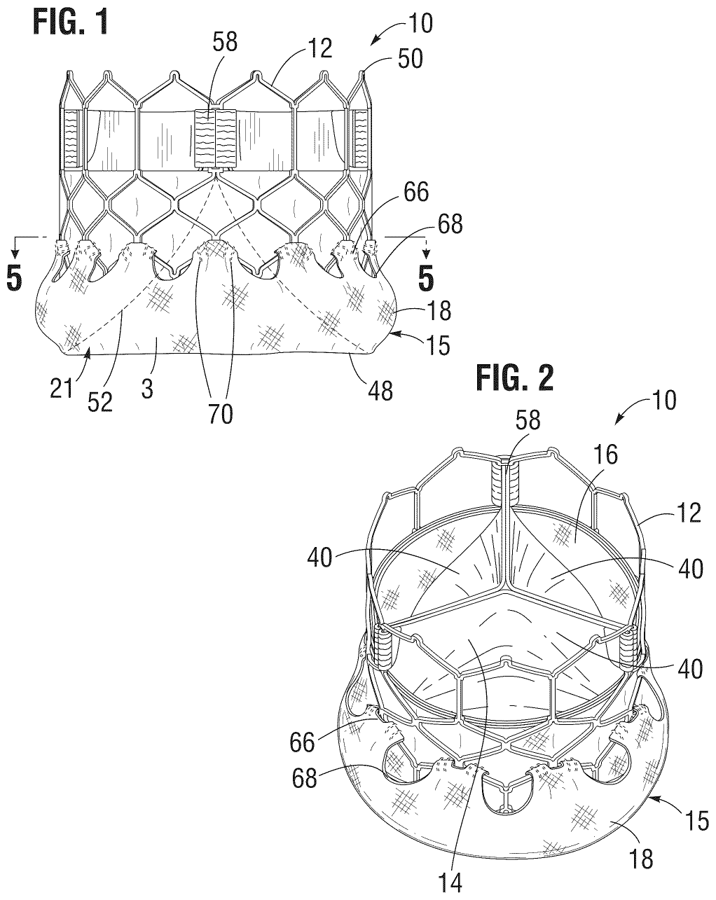

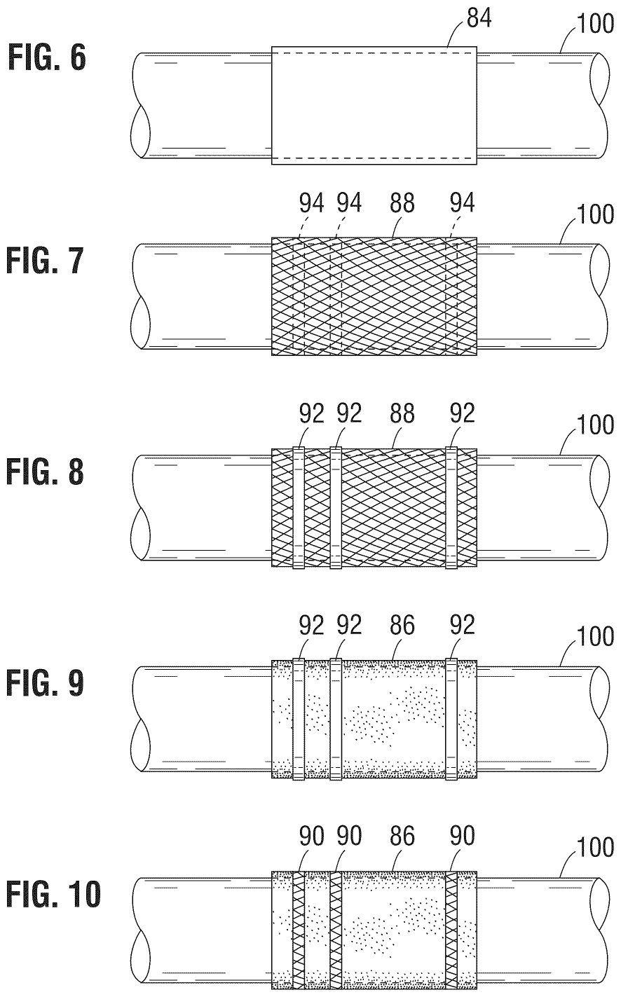

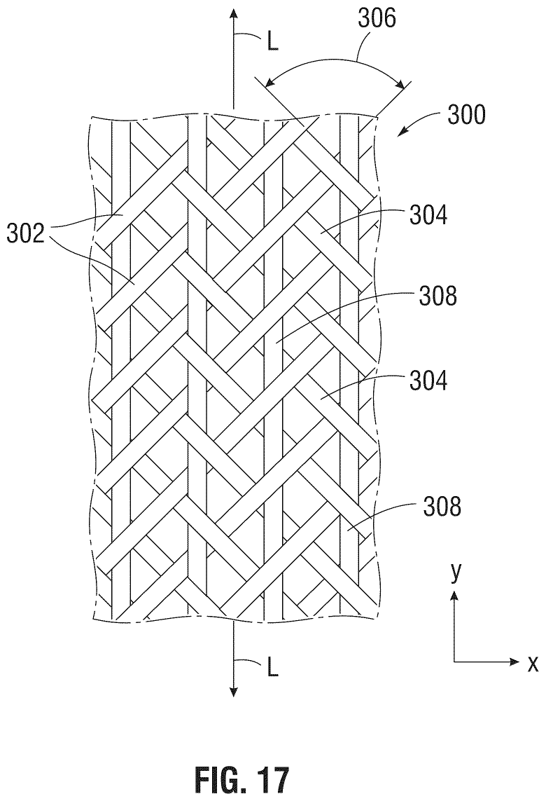

All features described herein are independent of one another and, except where structurally impossible, can be used in combination with any other feature described herein. For example, the exemplary prosthetic valve shown in FIGS. 1 and 2 can include any of the exemplary inner and outer skirts disclosed herein. In another example, the exemplary inner and outer skirts shown in FIGS. 1, 2, 5-15, and 20A-21 can include reinforcing layers having any of the exemplary weave patterns disclosed herein and/or illustrated in FIGS. 17-19J. In still another example, the exemplary inner and outer skirts 1, 2, 5-15, and 20A-21 can be manufactured via any of the techniques disclosed herein, such as via the technique illustrated in FIGS. 6-10.

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” or “enable” to describe the disclosed methods. These terms are high-level abstractions of the actual operations that are performed. The actual operations that correspond to these terms may vary depending on the particular implementation and are readily discernible by one of ordinary skill in the art.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used herein with reference to the prosthetic heart valve assembly and implantation and structures of the prosthetic heart valve, “proximal” refers to a position, direction, or portion of a component that is closer to the user and a handle of the delivery system or apparatus that is outside the patient, while “distal” refers to a position, direction, or portion of a component that is further away from the user and the handle, and closer to the implantation site. The terms “longitudinal” and “axial” refer to an axis extending in the proximal and distal directions, unless otherwise expressly defined.

The terms “axial direction,” “radial direction,” and “circumferential direction” have been used herein to describe the arrangement and assembly of components relative to the geometry of the frame of the prosthetic heart valve. Such terms have been used for convenient description, but the disclosed examples are not strictly limited to the description. In particular, where a component or action is described relative to a particular direction, directions parallel to the specified direction as well as minor deviations therefrom are included. Thus, a description of a component extending along an axial direction of the frame does not require the component to be aligned or overlapping with a center of the frame; rather, the component can extend substantially along a direction parallel to a central axis of the frame.

As used herein, the terms “integrally formed” and “unitary construction” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language. As used herein, “and/or” means “and” or “or,” as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,” “top,” “bottom,” “interior,” “exterior,” “left,” “right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

### Exemplary Prosthetic Valves

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

FIGS. 1 and 2 show two different views of a prosthetic valve 10, according to one example. The illustrated valve is adapted to be implanted in the native aortic annulus, although in other examples it can be adapted to be implanted in the other native annuluses of the heart. The valve 10 can have several main components: a stent or frame 12, a valvular structure 14, and a skirt assembly 15. The skirt assembly 15 can include an inner skirt 16, and optionally, an outer skirt 18.

The valvular structure 14 (or leaflet structure) can include three leaflets 40 (although a greater or fewer number of leaflets can be used), collectively forming the leaflet structure, which can be arranged to collapse in a tricuspid arrangement. The valvular structure 14 is configured to permit blood to flow through the prosthetic valve 10 in a direction from an inlet end 48 of the prosthetic valve to an outlet end 50 of the prosthetic valve, and to block the flow of blood through the prosthetic valve in a direction from the outlet end 50 to the inlet end 48.

Each leaflet 40 may have a curved, generally U-shaped inlet or cusp edge 52. In this manner, the inlet edge of the valvular structure 14 has an undulating, curved scalloped shape. By forming the leaflets with this scalloped geometry, stresses on the leaflets can be reduced, which in turn improves durability of the valve. Moreover, by virtue of the scalloped shape, folds and ripples at the belly of each leaflet (the central region of each leaflet), which can cause early calcification in those areas, can be eliminated or at least minimized. The scalloped geometry also reduces the amount of tissue material used to form leaflet structure, thereby allowing a smaller, more even crimped profile at the inflow end of the valve. The leaflets 40 can be formed of pericardial tissue (for example, bovine pericardial tissue), biocompatible synthetic materials, or various other suitable natural or synthetic materials as known in the art and described in U.S. Pat. No. 6,730,118, which is incorporated by reference herein.

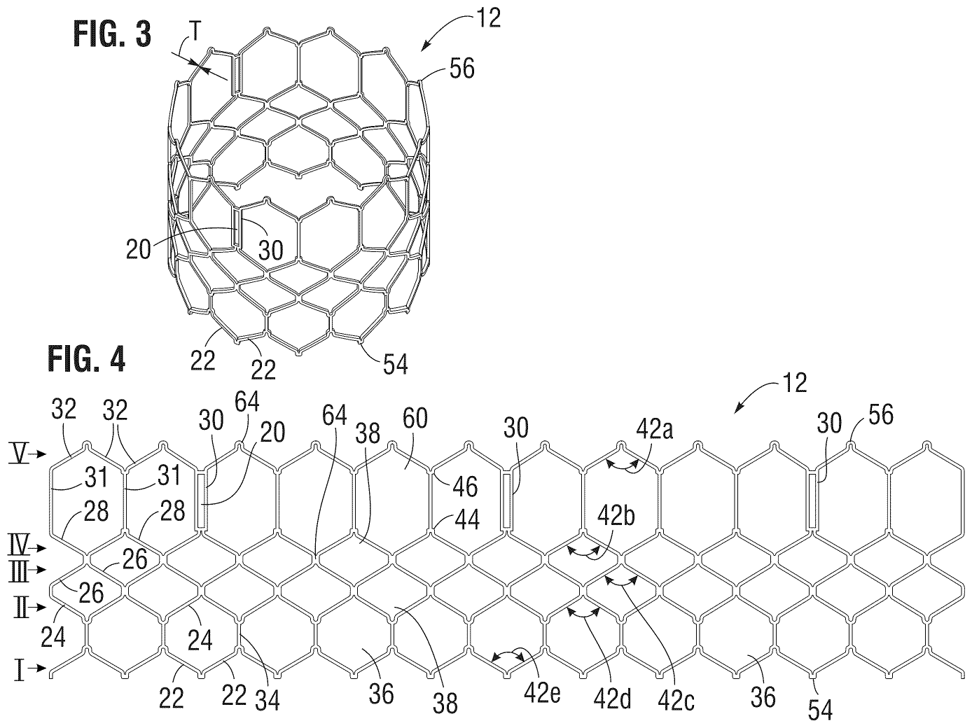

The bare frame 12 (with other elements of the prosthetic valve removed) is shown in FIG. 3. In the depicted example, the frame 12 has an annular shape defining an inlet end 54 and an outlet end 56 and includes a plurality of struts (or frame members). The frame 12 can be formed with a plurality of circumferentially spaced slots, or commissure windows, 20 (three in the illustrated example) that are adapted to mount the commissures 58 of the valvular structure 14 to the frame, as described more fully in U.S. Pat. No. 9,393,311, which is incorporated by reference herein.

The frame 12 can be made of any of various suitable plastically-expandable materials (for example, stainless steel, etc.) or self-expanding materials (for example, Nitinol) as known in the art. When constructed of an expandable material (for example, a plastically-expandable material), the frame 12 (and thus the valve 10) can be crimped to a radially compressed state on a delivery catheter and then expanded inside a patient by an inflatable balloon or another suitable expansion mechanism. When constructed of a self-expandable material, the frame 12 (and thus the valve 10) can be crimped to a radially compressed state and restrained in the compressed state by insertion into a sheath or equivalent mechanism of a delivery catheter. Once inside the body, the valve can be advanced from the delivery sheath, which allows the valve to expand to its functional size. An example of a prosthetic valve in a crimped or radially compressed state is illustrated and described in U.S. Pat. No. 11,013,600, which is incorporated by reference herein.

Suitable plastically-expandable materials that can be used to form the frame 12 include, without limitation, stainless steel, a nickel-based alloy (for example, a cobalt-chromium or a nickel-cobalt-chromium alloy), polymers, or combinations thereof. In particular examples, frame 12 is made of a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight. It has been found that the use of MP35N to form frame 12 may provide superior structural results over stainless steel. In particular, when MP35N is used as the frame material, less material is needed to achieve the same or better performance in radial and crush force resistance, fatigue resistances, and corrosion resistance. Moreover, since less material is required, the crimped profile of the frame can be reduced, thereby providing a lower profile valve assembly for percutaneous delivery to the treatment location in the body.

Referring to FIGS. 3 and 4, the frame 12 in the illustrated example includes a first, lower row I of angled struts 22 arranged end-to-end and extending circumferentially at the inflow end of the frame; a second row II of circumferentially extending, angled struts 24; a third row III of circumferentially extending, angled struts 26; a fourth row IV of circumferentially extending, angled struts 28; and a fifth row V of circumferentially extending, angled struts 32 at the outflow end 56 of the frame. A plurality of substantially straight axially extending struts 34 can be used to interconnect the struts 22 of the first row I with the struts 24 of the second row II. The fifth row V of angled struts 32 are connected to the fourth row IV of angled struts 28 by a plurality of axially extending window frame portions 30 (which define the commissure windows 20) and a plurality of axially extending struts 31. Each axial strut 31 and each frame portion 30 extends from a location defined by the convergence of the lower ends of two angled struts 32 to another location defined by the convergence of the upper ends of two angled struts 28.

Each commissure window frame portion 30 can mount a respective commissure 58 of the leaflet structure 14. Each frame portion 30 is secured at its upper and lower ends to the adjacent rows of struts to provide a robust configuration that enhances fatigue resistance under cyclic loading of the valve compared to known cantilevered struts for supporting the commissures of the leaflet structure. This configuration enables a reduction in the frame wall thickness to achieve a smaller crimped diameter of the prosthetic valve. In particular examples, the thickness T of the frame 12 (FIG. 3) measured between the inner diameter and outer diameter is about 0.48 mm or less.

The struts and frame portions of the frame collectively define a plurality of open cells of the frame. At the inflow end of the frame 12, struts 22, struts 24, and struts 34 define a lower row of cells defining openings 36. The second, third, and fourth rows of struts 24, 26, and 28 define two intermediate rows of cells defining openings 38. The fourth and fifth rows of struts 28 and 32, along with frame portions 30 and struts 31, define an upper row of cells defining openings 60. The openings 60 are relatively large and can be sized to allow portions of the leaflet structure 14 to protrude, or bulge, into and/or through the openings 60 when the frame 12 is crimped in order to minimize the crimping profile of the prosthetic valve.

As shown in FIG. 4, the lower end of the strut 31 is connected to two struts 28 at a node or junction 44, and the upper end of the strut 31 is connected to two struts 32 at a node or junction 46. The strut 31 can have a thickness that is less than the thicknesses of the junctions 44, 46. The junctions 44, 46, along with junctions 64, each of which links two adjacent struts 32, prevent full closure of openings 60 when the frame 12 is in a crimped state. Thus, the geometry of the struts 31, and junctions 44, 46 and 64 assists in creating enough space in openings 60 in the crimped state to allow portions of the leaflets to protrude (that is, bulge) outwardly through openings. This allows the valve to be crimped to a relatively smaller diameter than if all of the leaflet material is constrained within the crimped frame.

The frame 12 can be configured to prevent or at least minimize possible over-expansion of the valve at a predetermined balloon pressure, especially at the outflow end portion of the frame, which supports the leaflet structure 14. In one aspect, the frame can be configured to have relatively larger angles 42a, 42b, 42c, 42d, 42e between struts. The larger the angle, the greater the force required to open (expand) the frame. As such, the angles between the struts of the frame can be selected to limit radial expansion of the frame at a given opening pressure (for example, inflation pressure of the balloon). In particular examples, these angles are at least 110 degrees or greater when the frame is expanded to its functional size, and even more particularly these angles are at least 120 degrees or greater when the frame is expanded to its functional size. U.S. Pat. No. 9,393,110 (previously incorporated herein) further describes the frame 12, as well as other configurations for frames that can be incorporated in a prosthetic heart valve, such as the prosthetic valves 10 and 200 described herein.

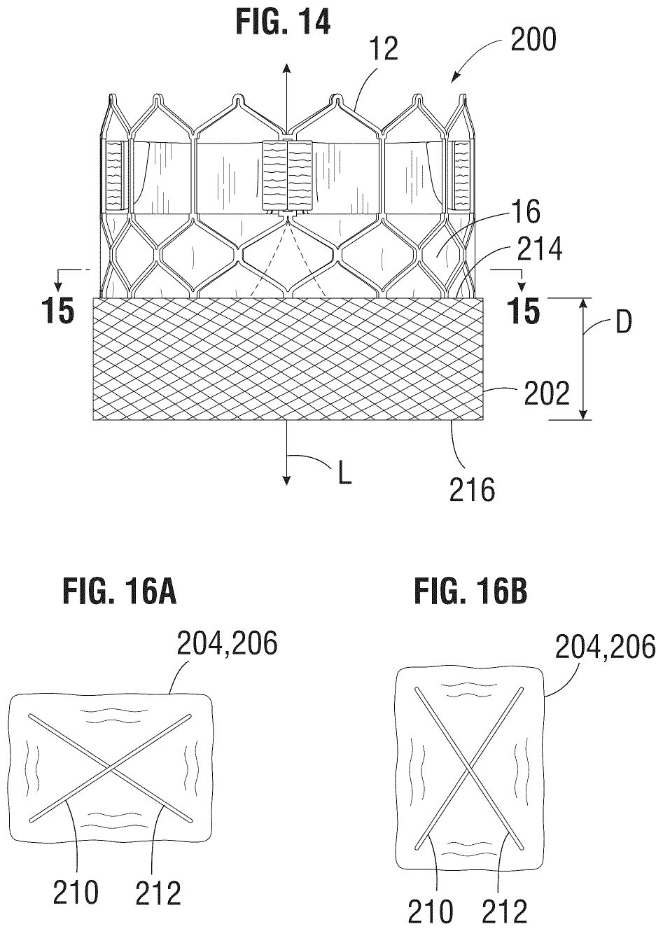

As shown in FIGS. 1, 2 and 5, the skirt assembly 15 can include an encapsulated inner skirt 16 that is located inside the frame 12 and an outer skirt 18 that is located outside the frame 12. The outer skirt 18 can include a plurality of circumferentially spaced apart extension portions or projections 66 and recesses 68 between adjacent projections formed along the outflow edge (the upper edge in the illustrated example) of the outer skirt. In other examples, the outer skirt 18 can have a straight outflow edge without any projections or recesses (similar to for example, the outer skirt 202 of FIG. 14).

The inflow (lower) and the outflow (upper) edges of the outer skirt 18 can be secured to the frame 12 and/or the inner skirt 16 by, for example, heat bonding, adhesive, and/or suturing. As shown in the illustrated example, the projections 66 along the outflow edge of the outer skirt 18 can be secured to struts of the frame with sutures 70 while the recesses 68 between adjacent projections can be left unattached to the frame 12 and the encapsulated inner skirt 16. The outer skirt 18 can function as a sealing member for the prosthetic valve 10 by sealing against the tissue of the native valve annulus, helping to reduce paravalvular leakage past the prosthetic valve 10.

In some examples, as shown in FIGS. 1 and 2, the outer skirt 18 can be configured to extend radially outward from the frame 12 when the prosthetic valve 10 is in a radially expanded configuration. Alternatively, the outer skirt 18 can be configured to form a snug fit with the frame 12 such that it lies against the outer surface of the frame 12 when the prosthetic valve 10 is in the radially expanded configuration (similar to for example, the outer skirt 202 of FIG. 14). The outer skirt 18 can be formed from any of various synthetic materials or natural tissue (for example, pericardial tissue). Suitable synthetic materials include any of various biocompatible fabrics (for example, PET fabric) or non-fabric films, including any of the materials disclosed below for the reinforcing layer 88 of the inner skirt 16. Further details of the outer skirt 18 are disclosed in U.S. Pat. No. 9,393,110 (previously incorporated herein).

As further shown in FIGS. 1 and 2, the encapsulated inner skirt 16 in the illustrated example extends from the inlet end 54 of the frame to the fourth row IV of angled struts 28. In other examples, the encapsulated inner skirt 16 can extend from the inlet end 54 of the frame to a location short of the fourth row IV of struts (for example, to the second row II or the third row III of struts), or the encapsulated inner skirt can extend the entire height of the frame 12 (for example, from the inlet end 54 to the outlet end 56). In alternative examples, the encapsulated inner skirt 16 can be positioned and/or sized to extend over a different portion of the frame 12 than the configuration shown in FIGS. 1 and 2. In some examples, the inflow end of the encapsulated inner skirt 16 can be axially spaced from the inlet end 54 of the frame 12.

Although the encapsulated inner skirt 16 is typically tubular or cylindrical in shape (forming a complete circle in a cross-sectional profile in a plane perpendicular to the longitudinal axis of the valve), the inner skirt 16 need not extend along the inner surface of the frame 12 in the circumferential direction through 360 degrees. In other words, the encapsulated inner skirt 16 can have a cross-sectional profile (in a plane perpendicular to the axis of the lumen of the valve) that is not a complete circle. The encapsulated inner skirt 16 can be initially formed as a flat strip, and then formed to the annular shape by coupling together opposing edge portions, for example, by sewing, thermal bonding, and/or adhesive. Alternatively, the encapsulated inner skirt 16 can be formed directly in an annular shape, for example, by constructing the encapsulated inner layer 16 on a cylindrically shaped mandrel as described below.

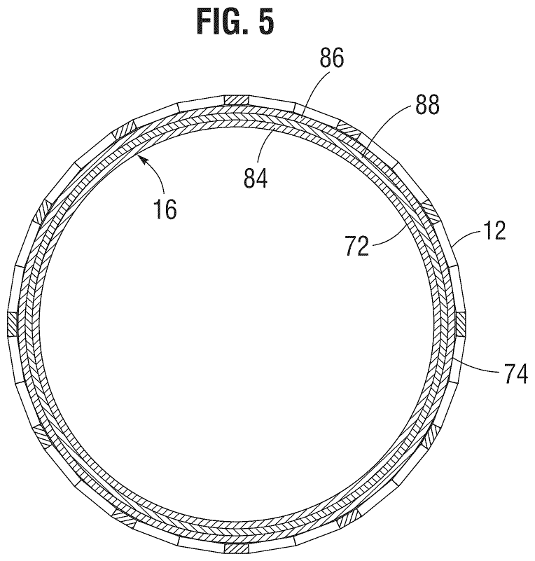

Referring to FIG. 5, the encapsulated inner skirt 16 has a first side 72 formed by a first encapsulating layer 84 and defining an inner surface of the inner skirt, and a second side 74 formed by a second encapsulating layer 86 and defining an outer surface of the inner skirt 16. As described more fully below, the frame-facing side 74 of the inner skirt 16 can have one or more windows or openings, through which an otherwise encapsulated fabric layer is exposed. Sutures can be threaded through the fabric layer of the inner skirt 16 at those windows to secure the inner skirt 16 to the frame 12. The outer skirt 18 is omitted in FIG. 5 for purposes of illustration.

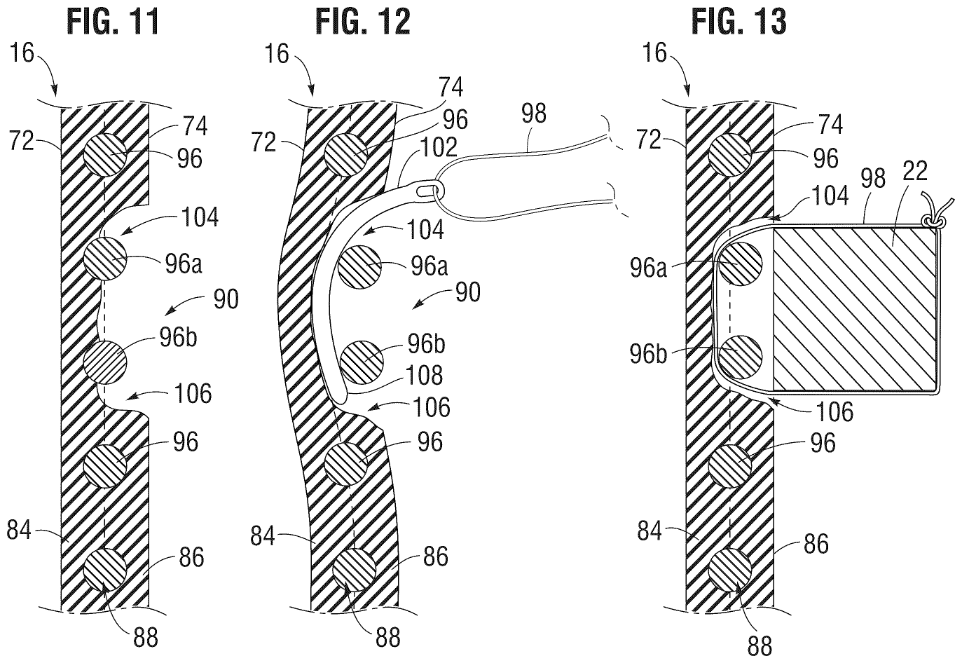

When the encapsulated inner skirt 16 is mounted to the frame 12, the first side 72 of the inner skirt 16 faces inwardly toward the leaflet structure 14 located interior of the prosthetic valve 10, and the second side 74 of the inner skirt 16 faces outwardly against the inner surface of the frame 12. In particular examples, the encapsulated inner skirt 16 can comprise a reinforcing layer 88 sandwiched between a first encapsulating layer 84 and a second encapsulating layer 86. In a representative example, the reinforcing layer 88 can be a fabric layer. The first and second encapsulating layers 84, 86 form the inner and outer layers, respectively, of the illustrated inner skirt 16. In particular examples, the inner surface of the reinforcing layer 88 can be completely covered by the first encapsulating layer 84 on the first side 72, and the outer surface of the reinforcing layer 88 is partially covered by the second encapsulating layer 86 on the second side 74, with the second encapsulating layer 86 defining one or more windows or openings 90 (see FIG. 10) that expose the reinforcing layer 88 on the second side 74.

The reinforcing layer 88 can strengthen the inner skirt 16 to resist tearing. It can also serve as an anchor layer for suturing the inner skirt 16 to the frame 12 and for supporting the cusp edge portions of the leaflets 40, as described more fully below. In addition, the reinforcing layer 88, in cooperation with the encapsulating layers 84, 86, can help decrease (or prevent) paravalvular leakage past the prosthetic valve 10 when in the expanded configuration.

In examples, the reinforcing layer 88 can comprise a fabric that is woven from various types of natural or synthetic fibers (or filaments, or yarns, or strands), including but are not limited to: gauze, PET fibers (for example, Dacron), polyester fibers, polyamide fibers, ultrahigh molecular weight polyethylene (UHMWPE) fibers, etc. In examples, the reinforcing layer 88 can have a woven, knitted, or braided pattern or structure similar to one or more of those discussed below with reference to FIGS. 17-19J (or another configuration). Additionally, the reinforcing layer 88 can be a woven, knitted, or braided structure formed from metal filaments or wires (for example, Nitinol, stainless steel, or titanium filaments or wires), glass filaments or wires, carbon filaments or wires, or ceramic (for example, alumina oxide) filaments or wires.

Alternatively, the reinforcing layer 88 can comprise filaments, fibers, yarns, or wires made of any of the materials described above, where filaments, fibers, yarns, or wires are not necessarily interwoven, braided, or knitted together. For example, the reinforcing layer 88 can comprise a layer of parallel filaments, fibers, yarns, or wires, or layers of filaments, fibers, yarns, or wires laid on top of each other. In certain examples, the reinforcing layer 88 can include any of various non-woven fabrics, such as felt. The thickness of the reinforcing layer 88 can vary, but can be less than 6 mil, and desirably less than 4 mil, and even more desirably about 2 mil.

Additionally or alternatively, the reinforcing layer 88 can include one or more layers or films formed from any of various semi-crystalline polymeric materials or thermoplastics having aligned or partially aligned (for example, parallel) molecular chains. Such materials can exhibit anisotropic mechanical properties, such as increased mechanical strength along the longitudinal direction of the molecular chains. Suitable semi-crystalline polymeric materials can include, for example, PTFE, PET, polypropylene, polyamide, polyetheretherketone (PEEK), etc., layers or films of which can be situated between and encapsulated by the encapsulating layers 84, 86, to reinforce the inner skirt 16. Unless otherwise specified, a fabric layer is described in the following description as an exemplary reinforcing layer for illustration purposes, while it is to be understood that a non-fabric layer having sufficiently high tensile strength can also be used as the reinforcing layer.

The encapsulating layers 84, 86 can be made of any suitable biocompatible material. Desirably, the encapsulating layers 84, 86 comprise a material that is relatively less abrasive than the fabric layer to reduce abrasion of the leaflets 40. The encapsulating layers 84, 86 can comprise, for example, a membrane or film formed from non-woven fibers or a non-fibrous material. The biocompatible material used to form the layers 84, 86 may be a non-absorbable polymeric material (that is, a material that does not dissolve once implanted in the body), and the material may be elastomeric.

In examples, any of the encapsulating layers 84, 86 can have a porous microstructure that promotes ingrowth of surrounding tissue to assist in securing the prosthetic valve 10 in a body lumen. In alternate examples, the encapsulating layers 84, 86 can be hermetic layers to prevent, or at least reduce or limit, the ingrowth of native tissue. As used herein, “hermetic layer” refers to a layer that is constructed such that, when implanted in a patient, ingrowth of cells into the layer is prevented or at least discouraged or limited. In some examples, such a hermetic layer is substantially nonporous or otherwise has pores therein of sufficiently small size to discourage cellular ingrowth. The size and characteristics of pores within the hermetic layer (for example, porosity, tortuosity) can be adapted to prevent cellular ingrowth, which may also depend on implant location (for example, pressure gradient, blood flow conditions), desired implant lifetime, hermetic layer thickness, and/or other factors. In some examples, a size of each pore in the hermetic layer can be, for example, 20 μm or less, 10 μm or less, or 5 μm or less. In some implementations, the hermetic layer may have a range of different pore sizes, and the distribution of pore sizes may be such that at least 90% of the pores have a size of 8 μm or less. Alternatively or additionally, in some implementations, the distribution of pore sizes in the hermetic layer may be such that at least 90% of the pores have a size of 20 μm or less, 10 μm or less, or 5 μm or less. Hermetic layers that can be utilized with the encapsulated skirts disclosed herein are further described in U.S. Provisional Patent Application Nos. 63/112,080 and 63/240,766, each of which is incorporated by reference herein.

Examples of encapsulating layer materials include, without limitation, cPTFE, unexpanded porous PTFE, polyester or expanded PTFE yarns, PTFE, ultrahigh molecular weight polyethylene (UHMWPE), other polyolefins, composite materials such as ePTFE with PTFE fibers, or UHMWPE film with embedded UHMWPE fibers, polyimides, silicones, polyurethane (PU), thermoplastic PU (TPU), other thermo plastic materials, hydrogels, fluoroethylpolypropylene (FEP), polypropylfluorinated amines (PFA), other related fluorinated polymers, or various combinations of the foregoing materials. In particular examples, the encapsulating layers 84, 86 can be formed from respective tubes made of a suitable polymeric material (for example, ePTFE tubes or UHMWPE tubes) that can be bonded to each other when subjected to heat treatment. In some examples, the encapsulating layers 84, 86 can be formed from the same type of materials, although different materials can be used to form the encapsulating layers depending on the particular application.

Microporous ePTFE tubes can be made by a number of well-known methods. Expanded PTFE is frequently produced by admixing particulate dry polytetrafluoroethylene resin with a liquid lubricant to form a viscous slurry. The mixture can be poured into a mold, typically a cylindrical mold, and compressed to form a cylindrical billet. The billet can then be ram extruded through an extrusion die into either tubular or sheet structures, termed extrudates in the art. The extrudates comprise an extruded PTFE-lubricant mixture called “wet PTFE.” Wet PTFE has a microstructure of coalesced, coherent PTFE resin particles in a highly crystalline state. Following extrusion, the wet PTFE can be heated to a temperature below the flash point of the lubricant to volatilize a major fraction of the lubricant from the PTFE extrudate. The resulting PTFE extrudate without a major fraction of lubricant is known in the art as dried PTFE. The dried PTFE can then be either uniaxially, biaxially, or radially expanded using appropriate mechanical apparatus known in the art. Expansion is typically carried out at an elevated temperature, for example, above room temperature but below 327° C., the crystalline melt point of PTFE. Uniaxial, biaxial, or radial expansion of the dried PTFE causes the coalesced, coherent PTFE resin to form fibrils emanating from nodes (regions of coalesced PTFE), with the fibrils oriented parallel to the axis of expansion. Once expanded, the dried PTFE is referred to as expanded PTFE (“ePTFE”) or microporous PTFE.

UHMWPE is made up of very long chains of polyethylene, with molecular weight numbering in the millions, usually between 2 to 6 million. It is highly resistant to corrosive chemicals, has extremely low moisture absorption and a very low coefficient of friction. It is self-lubricating and highly resistant to abrasion. UHMWPE is processed using compression molding, ram extrusion, gel spinning, and sintering. UHMWPE is available commercially as a powder, in sheets or rods, and as fibers.

The encapsulating layers 84, 86 can be formed by a number of means. For example, the encapsulating layers 84, 86 can be formed using an electrospinning process, which uses electric force to draw charged threads of polymer solutions or polymer melts up to fiber diameters in the order of some hundred nanometers. In another example, the encapsulating layers 84, 86 can be formed using a centrifugal spinning technique. In centrifugal spinning, the spinning fluid is placed in a rotating spinning head. When the rotating speed reaches a critical value, the centrifugal force overcomes the surface tension of the spinning fluid to eject a liquid jet from the nozzle tip of the spinning head. The jet then undergoes a stretching process and is eventually deposited on the collector, forming solidified nanofibers. In yet another example, the encapsulating layers 84, 86 can be formed using an atmospheric plasma spray (APS) technique, which is a special variation of the thermal spray process. APS uses an electric arc to ionize flowing process gases, the hot gas stream can be controlled to melt a very wide range of powder feedstock materials to apply high-quality coatings to a target object. In still other examples, the encapsulating layers 84, 86 can be formed using any other suitable method including, such as dip coating, spray coating, or melt-spinning. For example, any one of the encapsulating layers 84, 86 can be formed by dipping the fabric layer 88 in a liquefied polymeric material, and then allowing the liquefied polymeric material to cure.

FIGS. 6-10 show one exemplary process of forming the inner skirt 16 (and/or other encapsulated skirts described herein). Although the use of electrospinning is described below, it is exemplary in nature and is not intended as a limitation. It is to be understood that other processes for depositing a polymeric layer, such as centrifugal spinning, APS, dip coating, or others, can also be used.

First, as depicted in FIG. 6, a first encapsulating layer 84 comprising a first coating material can be deposited circumferentially around the outer surface of a cylindrically shaped mandrel 100 by means of electrospinning (or using other techniques). As known in the art, the electrospinning system can include a spinneret that is used to extrude a polymer solution or polymer melt to form fibers. To deposit the first encapsulating layer 84 over the mandrel 100, the electrospinning system can be configured to rotate the fiber-extruding spinneret in a circular motion around the mandrel 100. Alternatively, the fiber-extruding spinneret can be configured to be stationary while the mandrel 100 is placed in front of the spinneret and rotates about its longitudinal axis.

Second, as depicted in FIG. 7, the fabric layer 88 can be placed over the first encapsulating layer 84. The fabric layer 88 can be in the form of a sheet of fabric material that is tightly wrapped around the first encapsulating layer 84. For example, as described above, the fabric layer 88 can have a woven structure that includes threads of warp and weft fibers extending perpendicular to each other (such as those shown and described below with reference to FIGS. 17-19J).

In alternate examples, the fabric layer 88 may also be deposited over the first encapsulating layer 84. It should be understood that this method of construction is not limited to examples where the reinforcing layer is a plain weave fabric; this method can be used for forming a skirt wherein the reinforcing layer can take any of the forms disclosed herein. For example, the reinforcing layer 88 can be a pre-formed braided material or leno weaver material that is wrapped around the first encapsulating layer 84. In yet another example, layer 88 can be formed by braiding or leno weaving one or more yarns or filaments around the first encapsulating layer 84 to form a braided layer or leno weaver layer around the first encapsulating layer.

Third, as depicted in FIG. 8, one or more masks 92 can be placed over the fabric layer 88 at selected areas 94 of the fabric. Fourth, as depicted in FIG. 9, a second encapsulating layer 86 comprising of a second coating material can be deposited over the masked fabric layer 88 by means of electrospinning (or using other techniques). Next, as depicted in FIG. 10, the masks 92 are removed after the deposition of the second encapsulating layer 86. Accordingly, one or more windows 90 corresponding to the selected areas 94 are created such that the windows 90 expose the underlying fabric layer 88.

In the example shown FIGS. 8-9, the masks 92 can temporarily cover selected areas 94 of the fabric layer 88, and prevent those selected areas 94 from being deposited by the second coating material of the second encapsulating layer 86. Alternatively, the selected areas 94 may be functionally masked without the need of applying the physical masks 94. For example, the relevant movement and operation (for example, activation and/or deactivation) of the fiber-extruding spinneret with respect to the mandrel 100 may be programmed so that the second coating material of the second encapsulating layer 86 can only be deposited on portions of the fabric layer 88 that are outside the selected areas 94.

In the example depicted in FIGS. 8-9, three annular bands of masks 92 are shown, respectively corresponding to three selected areas 94 along the outer circumference of the fabric layer 88. As a result, three annular windows 90 are created after the removal of the masks 92, as depicted in FIG. 10. In other examples, any one of the masks 92 can have a non-annular shape, so that the corresponding selected area 94 and the resulting window 90 do not completely encircle the fabric layer 88. For example, any one of the masks 92 may have a customized shape at a customized location so as to create a customized window 90. In addition, although three windows 90 are shown in the illustrated example, the inner skirt can be formed with a fewer or greater number of windows and the windows can be positioned at any locations along the skirt. In examples, the inner skirt can be formed with one or more circumferentially extending rows of windows, with each row comprising of a plurality of circumferentially spaced-apart windows. In examples, one or more windows can be formed along the inlet and/or the outlet edge of the inner skirt.

Although not shown, it is to be understood that in each of the steps described above, an anchoring mechanism can be provided to temporarily secure the position of each layer. As a non-limiting example, layers of PTFE tape can be wrapped around one or both ends of the second encapsulating layer 86 to help secure the position of the second encapsulating layer 86 to the underlying layers of the assembly and to the mandrel 100 during subsequent processing.

In representative examples, the fabric layer 88 has a plurality of openings that allow the first encapsulating layer 84 and the second encapsulating layer 86 to fuse together through those openings. In examples, the openings in a fabric layer 88 may be created by weaving, braiding, or knitting fibers or yarns to form the fabric layer having a specified pattern (such as those shown and described below with reference to FIGS. 17-19J, or others). In another example, the fabric layer 88 may have a non-woven, porous structure with openings. In another example, such as when a non-woven fabric (for example, a felt) is used to form the fabric layer, openings in the fabric layer 88 can be formed by cutting (for example, laser cutting) openings in the fabric layer.

In examples, fusion between the first and second encapsulating layers 84, 86 through openings in the fabric layer 88 can occur simultaneously during the process of depositing the second encapsulating layer 86 over the masked fabric layer 88. As the second coating material extruded from the spinneret is deposited on the fabric layer 88 to form the second encapsulating layer 86, some of the second coating material may penetrate through those openings in the fabric layer 88, and fuse with the fibers in the first encapsulating layer 84.

In other examples, fusion between the first and second encapsulating layers 84, 86 may occur after the deposition of the second encapsulating layer 86 over the masked fabric layer 88. For example, the assembly shown in FIG. 10 can undergo an encapsulation process whereby the assembly is subjected to heat and/or pressure to cause the first and second encapsulating layers 84, 86 to bond to each other through the openings in the fabric layer 88. In addition, the fabric layer 88 may have an axial length that is shorter than the first and second encapsulating layers 84, 86 to facilitate bonding of the first and second encapsulating layers 84, 86 at their respective ends to encapsulate the fabric layer 88 therebetween. A similar encapsulation process is described in U.S. Pat. Nos. 10,045,846 and 10,232,564, which are each incorporated by reference herein.

In examples, ePTFE can be used as the first coating material for depositing the first encapsulating layer 84 and/or the second coating material for depositing the second encapsulating layer 86. Alternatively, other materials such as UHMWPE, polyurethane composite materials, or any other non-absorbable polymeric materials described above can be used. The inner skirt 16 desirably can have a laminate structure in which the fabric layer 88 is sandwiched between the two fused layers (that is, sandwiched between the fused first encapsulating layer 84 and second encapsulating layer 86). In some examples, the same material can be used for depositing the first and second encapsulating layers 84, 86. Due to the inter-layer fusion or bonding, the first and second encapsulating layers 84, 86 can be merged together, effectively creating a unitary structure (that is, there is no physical inter-layer boundary), in which the fabric layer 88 is encapsulated. The density of the first encapsulating layer 84 can be the same as or different from the density of the second encapsulating layer 86. In other examples, the first coating material used for depositing the first encapsulating layer 84 can be different from the second coating material used for depositing the second encapsulating layer 86.

After the first and second encapsulating layers 84, 86 are firmly fused together to encapsulate the fabric layer 88, the inner skirt 16 can be removed from the mandrel 100. One or both end portions of the inner skirt 16 can be trimmed to achieve the desired height of the inner skirt. The inner skirt 16 can then be mounted to the frame 12.

Although FIGS. 6-10 and the description above illustrate the process of forming an annularly shaped inner skirt 16, it is to be understood that the same process can be used to form the outer skirt 18. Further, as described above, the inner skirt 16 may be initially formed as a flat strip, and then formed to the annular shape by coupling together its two opposing edges. To form a flat strip, instead of using the cylindrically shaped mandrel 100, the first and second encapsulating layers 84, 86 as well as the fabric layer 88 may be constructed on a flat substrate (such as by for example, being formed on a flat metal plate having a compressible coating and pressed with a second flat metal plate, which is discussed further below).

Although the process described above uses masking to create the windows 90 on the second encapsulating layer 86 of the inner skirt 16, it is to be understood that other methods can be used to create those windows 90. For example, the second encapsulating layer 86 can be initially deposited over the entire surface of the fabric layer 88. Then selected areas 94 on the second encapsulating layer 86 can be located and removed, for example, by means of laser cutting, chemical erosion, or other means. As a result, windows 90 can be created at the selected areas 94 on the second encapsulating layer 86, exposing the underlying fabric layer 88 therein. In another example, the second encapsulating layer 86 can be pre-fabricated so that it is devoid of the second coating material in the selected areas 94. Then the pre-fabricated second encapsulating layer 86 can be wrapped around the fabric layer 88. As a result, the fabric layer 88 can be exposed through windows 90 created at the selected areas 94. Then, the assembly (the first encapsulating layer 84, the fabric layer 88, and the second encapsulating layer 86) can undergo the heat- and/or pressure-based encapsulation process as described above, causing the first and second encapsulating layers 84, 86 to bond to each other.

The inner skirt 16 can be sutured to the frame 12 at the locations of the windows 90. For example, the inner skirt 16 can be placed on the inside of frame 12. The positions of the windows 90 can be arranged so as to generally correspond to the first, third, and fourth rows of struts 22, 26 and 28, respectively, although other configurations can be used. The inner skirt 16 can also be secured to the struts of the first, third, and fourth rows of struts with sutures extending around the struts and through the fabric layer 88 at the locations of the windows 90, as further described below in connection with FIGS. 11-13. Since the windows in the illustrated example extend continuously around the entire circumference of the inner skirt, a continuous, circumferentially extending whip stitch can be formed along each row of struts and the fabric layer 88 at each window 90.

As described above, the windows 90 can be created at selected locations and can have any of various shapes, allowing the inner skirt to be sutured to the frame at different locations. For example, as noted above, the inner skirt can be formed with rows of circumferentially spaced-part windows, which can allow placement of individual sutures or stitching that does not extend continuously along an entire row of struts, such as at selected portions on the inner skirt that are more prone to tension or stress.

FIGS. 11-13 illustrate an exemplary method of suturing the inner skirt 16 to the frame 12. FIG. 11 shows a cross-sectional view of a portion of an inner skirt 16 having a first side 72 and a second side 74. As described above, the inner skirt 16 has an encapsulated fabric layer 88 sandwiched between the first encapsulating layer 84 on the first side 72 and the second encapsulating layer 86 on the second side 74. FIG. 11 also depicts a window 90 on the second encapsulating layer 86 of the inner skirt 16, exposing the underlying fabric layer 88. The inter-layer boundary (as indicated by the dashed line) may be absent when the same material is used to form the encapsulating layers 84, 86, which can be fused or bonded together to form a single, unitary structure encapsulating the fabric. In the depicted example, the fabric layer 88 is shown to have a woven structure comprising woven filaments, fibers, or yarns 96. The woven filaments 96 desirably have sufficient strength to serve as anchors to retain sutures 98, as described below.

FIG. 12 schematically shows how a suture 98 may be threaded between the fabric layer 88 and the first encapsulating layer 84 through the window 90. In the depicted example, the suture 98 is attached to a needle 102. The tip 108 of the needle 102 may be blunted to avoid piercing the first encapsulating layer 84. By sliding the needle 102 from the second side 74 and through the window 90 while applying light force at the needle tip 108, the first encapsulating layer 84 can be slightly pushed away from the fabric layer 88, thus creating a space for the needle 102 to be inserted between the first encapsulating layer 84 and the fabric layer 88. As shown, the needle 102 and the attached suture 98 can slide into the fabric layer 88 from a first end 104 of the window 90, pass behind one or more filaments 96 that are exposed by the window 90 (for example, 96a and 96b), and then slide out of fabric layer 88 at a second end 106 of the window 80. In this manner, the suture 98 does not extend through the entire thickness of the inner skirt 16. In some examples, the first encapsulating layer 84 may not separate from the fabric layer 88 by insertion of the needle 102 (as depicted in FIG. 12), in which case the needle 102 and the attached suture 98 may be threaded partially through the thickness of the first encapsulating layer 84 but does not extend through the entire thickness of the inner skirt.

FIG. 13 schematically illustrates suturing of the fabric layer 88 of the inner skirt 16 to an adjacent strut 22 of the frame 12. Desirably, the first side 72 of the inner skirt 16 faces inwardly toward the leaflet structure 14 located interior of the prosthetic valve 10, and the second side 74 of the inner skirt 16 faces outwardly against the frame 12. By threading the needle 102 and the attached suture 98 between the fabric layer 88 and the first encapsulating layer 84 through the window 90, the woven filaments 96 between the first and second ends 104, 106 of the window 80 (for example, 96a and 96b) can collectively serve as anchors to hold the suture 98. The suture 98 can then be wrapped around the adjacent strut 22, thus securing those woven filaments (for example, 96a and 96b) to the adjacent strut 22. Accordingly, the inner skirt 16 can be securely attached to the frame 12. FIG. 13 depicts a strut 22 for purposes of illustration. It should be understood that the inner skirt 16 can be sutured to other struts of the frame (for example, 26, 28, 32) in a similar manner.

As described above, the fabric layer 88 may also have a non-woven structure that has no distinct woven threads. In such case, the suture 98 may be attached to a needle that has a pointed tip. The needle can be used to pierce the fabric layer 88 and thread the suture 98 through the fabric layer. In this manner, the portion of the fabric layer 88 that is between the first and second ends 104, 106 of the window 90 can function as an anchor to hold the suture 98, which in turns secures that portion of the fabric to the adjacent strut 22. Accordingly, the inner skirt 16 can be securely attached to the frame 12.

Because the suture 98 is routed between the first encapsulating layer 84 and the fabric layer 88, it is not exposed on the first side 72 of the inner skirt 16. In other words, the suture 98 is covered by the first encapsulating layer 84. The inner surface of the fabric layer is also covered by the first encapsulating layer. Therefore, abrasion of leaflets 40 due to repetitive contact between the leaflets 40 and inner skirt 16 and between the leaflets 40 and the sutures 98 during working cycles of the prosthetic valve 10 can be avoided. Desirably, the inner skirt 16 is sutured to the frame 12 only at the one or more windows 90 on the second encapsulating layer 86, so that contact between the moveable portions of the leaflets 40 and the sutures 98 can be avoided. Additionally, the first encapsulating layer 84 desirably covers the entire extent of the inner surface of the fabric layer, or at least the portions of the fabric layer that would otherwise contact the moveable portions of the leaflet during working cycles of the prosthetic valve. In some examples, sutures 98 may pass through the entire thickness of the inner skirt, such as at locations on the inner skirt that would not come in contact with moveable portions of the leaflets.

As noted above, the leaflets 40 can be secured to one another at their adjacent sides to form commissures 58. Each commissure 58 can be secured to a corresponding commissure window 20 of the frame 12, as described in U.S. Patent Publication No. 2012/0123529. The inflow or cusp edges 52 of the leaflets 40 can be sutured to the inner skirt 16 along a suture line that tracks the curvature of the scalloped inflow edge of the leaflet structure. The fabric layer 88 can provide the strength required to retain the sutures. Any suitable suture, such as an Ethibond suture, can be used to secure the leaflets 40 to the fabric layer 88 of the inner skirt.

In some examples, the inflow edges 52 of the leaflets 40 are secured to the inner skirt 16 prior to mounting the inner skirt 16 to the frame. After securing the leaflets 40 to the inner skirt 16, the inner skirt is then secured to the frame as described above and the commissures 58 of the leaflets are mounted to the frame. In other examples, the inner skirt 16 can be mounted to the frame without the leaflets, after which the inflow edges 52 of the leaflets are then secured to the inner skirt.

In certain examples, the inflow edges 52 of the leaflets 40 can be secured to the inner skirt via a thin PET reinforcing strip (not shown), as disclosed in U.S. Pat. No. 7,993,394, which is incorporated herein by reference. As described in U.S. Pat. No. 7,993,394, the reinforcing strip can be sutured to the inflow edges of the leaflets. The reinforcing strip and the lower edges of the leaflets can then be sutured to the inner skirt 16. The reinforcing strip desirably is secured to the inner surfaces of the leaflets 40 such that the inflow edges 52 of the leaflets become sandwiched between the reinforcing strip and the inner skirt when the leaflets and the reinforcing strip are secured to the inner skirt. The reinforcing strip can enable a secure suturing and protects the pericardial tissue of the leaflet structure from tears.

In examples, the outer skirt 18 can be constructed in a similar manner as the inner skirt 16. That is, the outer skirt 18 can also have a reinforcing layer encapsulated by interior and exterior encapsulating layers (such as a structure similar to the fabric layer 88 sandwiched between encapsulating layers 84, 86). Windows (similar to windows 90) can be created on one of the encapsulating layers of the outer skirt 18. Because the outer skirt 18 is attached to the outside of the frame 12, the outer layer 18 is desirably arranged so that the frame 12 faces the side of the outer skirt 18 that has the windows. In such arrangements, the outer skirt 18 can be attached to the frame 12 by suturing the encapsulated fabric layer to the frame 12 through the frame-facing windows.

The reinforcing layer in the outer skirt can be a fabric comprised of one or more of the materials discussed above with reference to the reinforcing layer 88. Further, the reinforcing layer in the outer skirt can have a woven, knitted, or braided pattern or structure, such as those discussed below with reference to FIGS. 17-19J (or another configuration). Furthermore, the encapsulating layers for the outer skirt 18 can be manufactured via the methods described above with reference to the encapsulated inner skirt 16 and/or can be comprised of the materials discussed above with reference to the encapsulating layers 84, 86.

In another example, the outer skirt 18 can include a fabric layer coated with only one encapsulating layer. While attaching the outer skirt 18 to the frame 12, the outer skirt 18 can be arranged so that the uncoated (non-encapsulated) side of the fabric layer faces inwardly toward the frame 12, so that the outer skirt 18 can be attached to the frame 12 by suturing the exposed fabric layer to the frame 12. Alternatively, the uncoated side of the fabric layer can face outwardly from the frame 12.

In another example, the outer skirt 18 can comprise only a fabric layer lacking any encapsulating layers. As such, the outer skirt 18 can be directly sutured to the frame 12. Because the sutures on the outer skirt 18 are not subject to repetitive contact by the moving leaflets 40, abrasion of the leaflets due to the sutures on the outer skirt 18 may be less of a concern than the sutures on the inner skirt 16. By eliminating one or both encapsulating layers, the outer layer 18 may have a relatively thinner construction, thereby reducing the overall profile of the prosthetic valve 10 when crimped into a radially compressed state.

FIG. 14 illustrates a prosthetic valve 200, according to another example. The prosthetic valve 200 can have a similar construction and components as the prosthetic valve 10 of FIGS. 1 and 2, except that the prosthetic valve 200 includes an encapsulated outer skirt 202. Common components in FIGS. 1 and 2 and FIG. 14 are designated with the same reference numbers and are not described further.

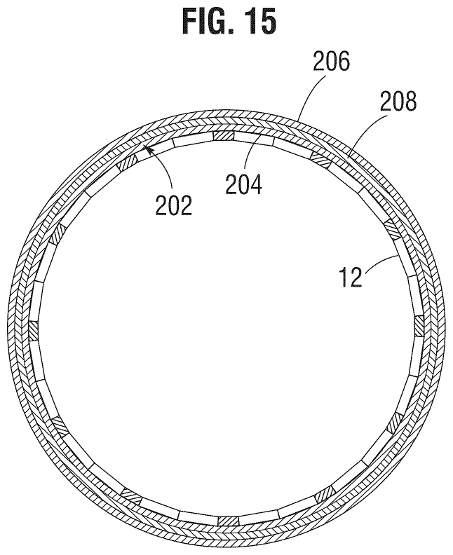

As can be seen in FIGS. 14 and 15, the encapsulated outer skirt 202 can be similar in construction to the encapsulated inner skirt 16. Specifically, the outer skirt 202 includes an inner (first) encapsulating layer 204, an outer (second) encapsulating layer 206, and a reinforcing layer 208 disposed between the encapsulating layers 204, 206. The outer skirt is mounted on the outside of the frame 12. In examples, the skirt 202 can be formed and mounted to the frame 12 using one or more of the techniques described above in connection with the encapsulated skirt 16, and can include materials in the reinforcing layer and/or the encapsulating layers described above with reference to the encapsulated skirt 16. Further, in examples, the reinforcing layers 88 and/or 208 can have a woven, knitted, or braided pattern or configuration similar to one or more of those discussed below with reference to FIGS. 17-19J (or another pattern or configuration).

FIG. 15 shows cross-sectional view of the frame 12 and the outer skirt 202 with the other components of the prosthetic valve removed for purposes of illustration. The outer skirt 202 can be configured to form a snug fit with the frame 12 such that it lies against the outer surface of the frame 12 when the prosthetic valve 200 is in the radially expanded configuration (shown in FIGS. 14-15).

It will be appreciated that the prosthetic valve 200 can optionally include an encapsulated inner skirt, similar to the inner skirt 16, or can include a non-encapsulated inner skirt. In alternate examples, the prosthetic valve 200 (and/or the prosthetic valve 10) can include other configurations for inner and outer skirts. For example, the inner and outer skirts can be a continuous layer extending over portions or over an entirety of the inner and/or outer surfaces of the frame. Such configurations are shown and described in U.S. Provisional Patent Application Nos. 63/112,080 and 63/240,766, previously incorporated herein.

It will be further appreciated that when the prosthetic valves 10 and 200 are crimped to a radially compressed state for delivery into a patient's body (for example, on a balloon of a delivery apparatus, or within a sheath delivery apparatus for a self-expanding valve), the frame 12 is elongated in the direction of the frame's longitudinal axis L. When the prosthetic valve is radially expanded from the radially compressed state to a radially expanded state, the frame 12 foreshortens axially along the axis L. Thus, it is desirable that encapsulated skirts (such as for example, the encapsulated inner skirt 16 and the encapsulated outer skirt 202) exhibit sufficient elongation or stretchability in the axial direction so as not to inhibit elongation of the frame 12 during crimping of the prosthetic valve.

To such ends, in particular examples, the reinforcement layers 88, 208 can comprise yarns, filaments, fibers, or wires (in a woven, braided or knitted structure or pattern) that are non-parallel and non-perpendicular upper and lower edges of the skirt. In examples, the reinforcement layers 88, 208 can include yarns, filaments, fibers, and/or wires that are non-parallel and non-perpendicular upper and lower edges, respectively, of the inner skirt 16 and the outer skirt 202. Stated differently, the yarns, filaments, fibers, or wires extend at angles greater than 0 degrees and less than 90 degrees (such as for example, 45 degrees) relative to the longitudinal axis L of the frame 12.

In one specific example shown in FIGS. 16A and 16B, the reinforcing layer 208 can comprise a fabric having a first set of yarns 210 interwoven with a second set of yarns 212, wherein the yarns 210, 212 are non-parallel and non-perpendicular to the upper and lower edges 214, 216 of the encapsulated outer skirt 202. Stated differently, the yarns 210, 212 extend at angles greater than 0 degrees and less than 90 degrees relative to the longitudinal axis L of the frame 12.

In some examples, in a relaxed state of the skirt, the yarns 210, 212 extend at angles in the range of 20 degrees to 70 degrees relative to the upper and lower edges 214, 216, or more narrowly in the range of 30 degrees to 60 degrees relative to the upper and lower edges 214, 216, or even more narrowly the range of 40 degrees to 50 degrees relative to the upper and lower edges 214, 216. In a specific example, the yarns 210, 212 extends at 45 degrees relative to the upper and lower edges 214, 216 and a longitudinal axis L of the prosthetic valve 200.

In examples, the yarns 210, 212 can be approximately parallel to some of the struts of the frame to which the outer skirt is connected when the prosthetic valve is in the expanded state. In particular examples, the skirt 202 is sutured to one or more of the angled struts 22, 24, 26, and 28 (see FIG. 4). The frame struts to which the outer skirt is connected to can be referred to as “skirt-supporting struts.” Typically, though not necessarily, the skirt-supporting struts are oriented at 45-degree angles relative to the longitudinal axis L when the frame is in the radially expanded state. However, in other examples, the skirt-supporting struts and the yarns 210, 212 can be greater or less than 45-degrees relative to the longitudinal axis L, depending on the particular frame configuration.

The reinforcing layer 208 can be formed by weaving the yarns at the selected angle (for example, 45 degrees) relative to the upper and lower edges of the fabric. Alternatively, the reinforcing layer 208 can be diagonally cut from a vertically woven fabric (where the yarns extend perpendicular to the edges of the material) such that the fibers extend at a selected angles (for example, 45 degrees) relative to the cut upper and lower edges of the fabric. The yarns 210, 212 can comprise multi-filament yarns (yarns comprising plural fibers or filaments) or mono-filament yarns (yarns comprising single fibers or filaments).

In examples, the frame 12 can be partially crimped during assembly of the prosthetic valve. The encapsulated outer skirt 202 can then be mounted to the frame in the partially crimped state, such as via any of the techniques described herein, so that the skirt-supporting struts are approximately parallel to the yarns 210, 212 in the partially crimped state. In other examples, the outer skirt 202 can be formed on or mounted to the frame 12 in a radially expanded state, and the outer skirt 202 and the frame 12 can be crimped together or concurrently.

Due to the orientation of the yarns relative to the upper and lower edges, the fabric layer can undergo greater elongation in the axial direction (that is, in a direction from the upper edge 214 to the lower edge 216 parallel to axis L). Thus, when the metal frame 12 is crimped, the skirt 202 can elongate in the axial direction along with the frame and therefore provides a more uniform and predictable crimping profile. Each cell of the metal frame in the illustrated example includes at least four angled struts (for example, struts 22, 24, 26) that can be moved to towards alignment with the axial direction (that is, the angled struts become more aligned with the length of the frame). The angled struts of each cell function as a mechanism for moving the yarns 210, 212 of the reinforcing layer 208 in the same manner and/or direction of the struts, allowing the skirt 202 to elongate along the length of the struts. This can allow for greater elongation of the skirt and avoids undesirable deformation of the struts when the prosthetic valve is crimped. As described above, the encapsulating layers 204, 206 can be made of any various elastomers described above in connection with layers 84 and 86, such as silicone or polyurethane or thermoplastics, that can stretch axially when the prosthetic valve is crimped and the frame is elongated.

The movement of the yarns 210, 212 during crimping is illustrated in FIGS. 16A and 16B. FIG. 16A shows portions of two interwoven yarns 210, 212 when the outer skirt 202 is in a relaxed state, corresponding to the radially expanded state of the prosthetic valve 200. FIG. 16B shows the yarns 210, 212 when the outer skirt 202 is in an axially stretched or elongated state, corresponding to the radially compressed state of the prosthetic valve after crimping. As shown, the yarns 210, 212 move from a 45-degree angle orientation relative to the longitudinal axis L toward an orientation where the yarns are closer to parallel with the longitudinal axis L.

In examples, the spacing between the woven (or braided or knitted) yarns can be increased to facilitate elongation of the encapsulated outer skirt 202 in the axial direction. For example, in particular examples, the reinforcing layer 208 can have a weave density less than 150 ppi (picks per inch), less than 100 ppi, less than 70 ppi, or 50 ppi or less. In certain examples, the reinforcing layer 208 can have a weave density of about 30 to about 50 ppi, as compared to a known fabric skirts that have a weave density of about 150 to 160 ppi. This construction can allow the skirt 202 to stretch or elongate axially up to at least 40% of its initial length D when the prosthetic valve is radially compressed (the initial length D being the distance between the upper and lower edges 214, 216 of the skirt when the prosthetic valve is in the radially expanded state). At such low weave densities, textiles with interlacing yarns (for example, woven, braided or knitted structures) can be unstable and tend to unravel. Advantageously, the encapsulating layers 204, 206 encapsulate the yarns and serve as a support mechanism that prevents unraveling of the relatively loose weave. Moreover, during the assembly process described above in connection with FIGS. 6-10, the first encapsulating layer (layer 84 in FIGS. 6-10) can adhere to the textile reinforcing layer (layer 88), which helps stabilize the textile and can avoid unraveling until the second encapsulating layer (layer 86) is formed or placed over the textile reinforcing layer. In addition, as noted above, the textile reinforcing layer in some examples can be braided around the first encapsulating layer supported on a mandrel 100, which further helps stabilize the reinforcing layer 208.

In certain examples, the yarns 210, 212 can have about 10 to about 50 filaments per yarn, with 20 filaments per yarn being a specific example. The filaments of the yarns 210, 212 can have a thickness in the range of about 8 μm to about 20 μm, with 10 μm being a specific example. The filaments of the yarns 210, 212 can be made of TPU, PET, or UHMWPE, although it will be appreciated that the filaments can be made of any of the materials described above in connection with the reinforcing layer 88.

In particular examples, the yarns 210, 212 can be texturized to increase the elasticity of the skirt in the axial direction. For example, the yarns can be bulked, wherein for example, the yarns are twisted or coiled, heat set, and untwisted or uncoiled such that the yarns retain their deformed (for example, twisted or coiled) shape in the relaxed or non-stretched configuration. When the prosthetic valve is radially compressed and the skirt 202 is stretched axially, the yarns 210, 212 can straighten from their deformed (for example, twisted or coiled) state to increase the elasticity and elongation of the skirt. Fabrics comprising texturized yarns are further disclosed in U.S. Publication No. 2018/0206982, which is incorporated by reference herein. Since the yarns 210, 212 are embedded within elastomeric encapsulating layers 204, 206, the encapsulating layers can stretch axially to accommodate the straightening of the yarns 210, 212. Utilizing such texturized yarns 210, 212 can allow the skirt 202 to stretch axially greater than 40% of its initially length D when the prosthetic valve is radially compressed.

It will be appreciated that the examples described above and shown in FIGS. 16A and 16B with respect to the reinforcing layer 208 of the encapsulated outer skirt 202 can be applied to the reinforcing layer 88 of the encapsulated inner skirt 16, or other skirt configurations, such as those described in U.S. Provisional Patent Application Nos. 63/112,080 and 63/240,766, previously incorporated by reference herein.

### Exemplary Reinforcing Layer Weave Patterns

FIG. 17 shows an exemplary section of a braided layer 300 that can be used as a reinforcing layer, such as the reinforcing layers 88, 208, in an encapsulated skirt. In FIG. 17, the x-axis represents the circumferential direction of an encapsulated skirt and the y-axis represents the axial direction of the encapsulated skirt. The braided layer 300 is constructed of first and second sets of yarns 302, 304 braided together, such as in a biaxial braid. FIG. 17 shows the braided layer 300 when the prosthetic valve is radially expanded. The illustrated example can be a relaxed state of the braided layer, meaning that the braided layer is not stretched or elongated in in the axial direction. As shown, the yarns 302, 304 can be oriented at approximately 45-degree angles with respect to the longitudinal axis L of a prosthetic valve and can form a braid angle 306 of 90 degrees. In some examples, the yarns 302, 304 can extend parallel to the struts of the frame 12 when the frame is in the radially expanded state.

To help stabilize and/or increase tensile strength of the braid, the layer 300 can include a third set of axially extending yarns 308 braided together with yarns 302, 304 to form a triaxial braid. The yarns 308 can be made of an elastomer (for example, PU or TPU) that can stretch axially when the prosthetic valve is radially compressed. In lieu of or in addition to forming the yarns 308 from an elastomer, the yarns 308 can be texturized as disclosed above to increase the elasticity of the skirt in the axial direction. The yarns 302, 304 can be made of the same material as the yarns 308 or a different material.

Further, the braided layer 300 can be a hybrid textile (for example, a woven, braided, or knitted structure) that incorporates yarns or filaments of different materials with different material properties. For example, one or more yarns of the textile can be relatively non-elastic or less elastic than one or more other yarns of the textile. In one implementation, for example, the one or more relatively less elastic yarns can be made of PET and the one or more relatively more elastic yarns can be made of PU, TPU, or other elastomers. The less elastic yarns can be selectively positioned within the textile (according to a predefined pattern) to strengthen the textile while the more elastic yarns can be selectively positioned (according to a predefined pattern) to promote elasticity in a given direction (for example, in the axial direction). Specifically, in the example of FIG. 17, the yarns 302, 304 can be made of PET and the yarns 308 can be made of PU or TPU.

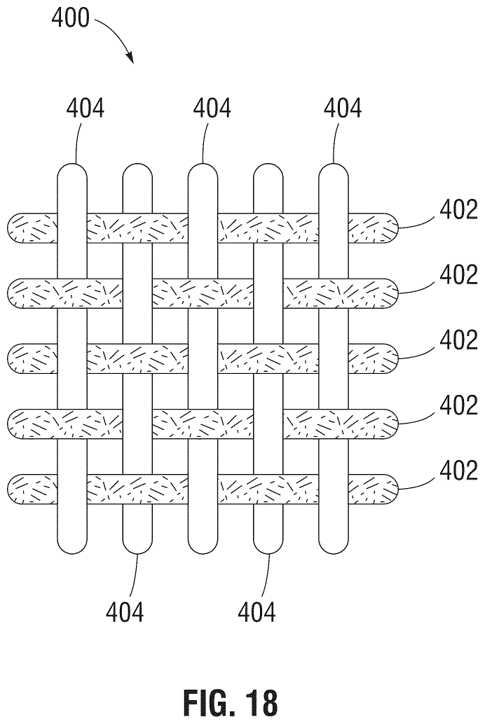

In other examples, a reinforcing layer (such as the reinforcing layers 88, 208) can be constructed from a fabric layer 400 having a woven structure (for example, plain weave, including variants). Plain weave (also called tabby weave) is a weave in which every weft yarn alternately passes over and under the warp yarns, forming a crisscross pattern. The warp yarns lie parallel to each other across the warp. FIG. 18 illustrates a plain weave including weft yarns 402 crisscrossing warp yarns 404. Plain weave produces the greatest number of intersections per unit space compared to other weave patterns, which can result in a strong and durable fabric. Variations of the plain weave include the rib weave, with either warp or weft yarns heavier, and the basket weave, in which two or more weft yarns pass alternately over and under two or more warp yarns. The high number of intersections can also allow the fabric to be relatively thin while being strong, allowing the outer skirt 202 to be sutured to the frame 12 and allowing the prosthetic heart valve to be crimped without tearing the reinforcing layer 208.

As described above, the weft yarns 402 and the warp yarns 404 can each be disposed at a non-parallel and non-perpendicular orientation relative upper and lower edges 214, 216, respectively, of the skirt 202. In examples, the weft yarns 402 and the warp yarns 404 can be moved from an approximately 45-degree angle orientation relative to the longitudinal axis L (when the outer skirt 202 is in a relaxed state, corresponding to the radially expanded state of the prosthetic valve 200) toward an orientation where the yarns 402, 404 are closer to parallel with the longitudinal axis L (when the outer skirt 202 is in an axially stretched or elongated state, corresponding to the radially compressed state of the prosthetic valve, such as after crimping). Further, the weft yarns 402 and the warp yarns 404 can have a material composition similar to those described above.

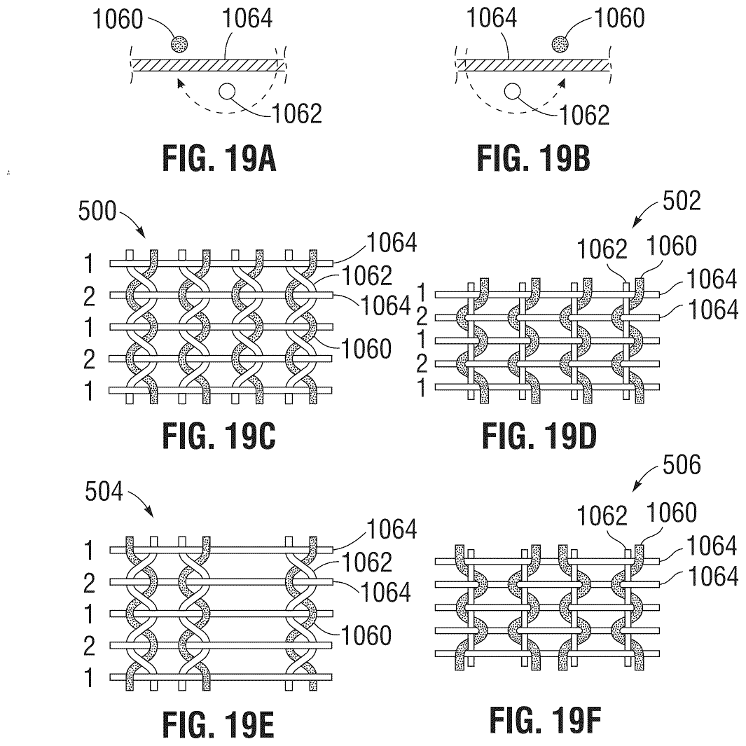

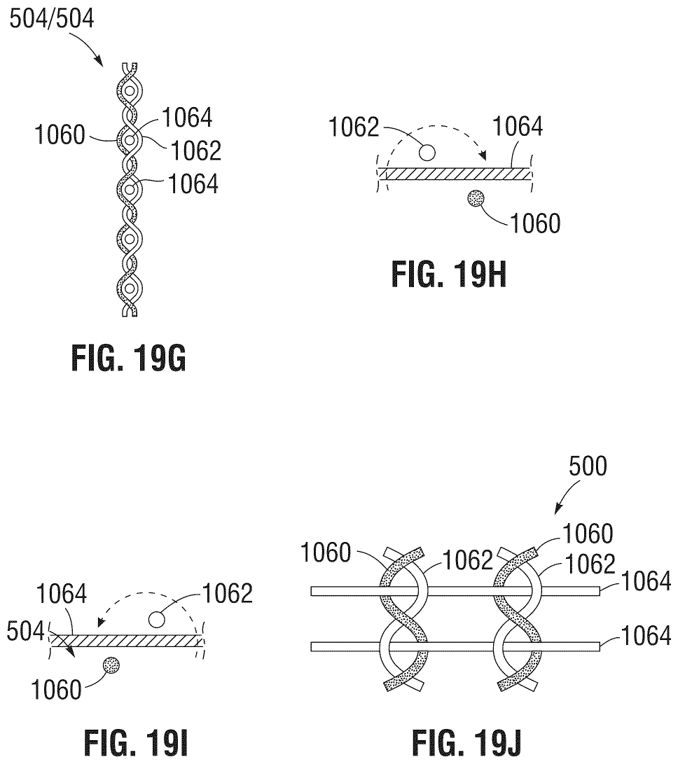

In other examples, a reinforcing layer (such as the reinforcing layers 88, 208) can be a fabric that includes one or more leno yarns woven into the fabric to increase the stability of the fabric and enable a looser weave or decreased weave density. Various leno weave patterns can be used to weave the leno yarns into the reinforcing layers 88, 208. Examples of half-leno weaves, full-leno weaves, and associated weaving techniques which may be utilized to produce a reinforcing layer are illustrated in FIGS. 19A-19J.

Specifically, FIG. 19A is a cross-sectional view illustrating a shed (for example, the temporary separation of warp strands/yarns to form upper and lower warp strands/yarns) in which a leno yarn, “leno end,” or “crossing end” 1060 forms the top shed on the left of the figure above a weft strand/yarn 1064 and a standard warp strand/yarn 1062 forms the bottom shed. FIG. 19B illustrates a successive shed in which the leno strand/yarn 1060 forms the top shed on the right of the standard warp strand/yarn 1062. In FIGS. 19A and 19B, the leno strand/yarn 1060 can cross under the standard strand/yarn 1062 in a pattern known as bottom douping. Alternatively, the leno strand/yarn 1060 can cross over the standard strand/yarn 1062, known as top douping, as in FIGS. 19H and 19I.

FIG. 19C illustrates an exemplary leno weave interlacing pattern 500 produced when one warp beam is used on a loom and the distortion or tension of the leno strands/yarns 1060 and the standard strands/yarns 1062 is equal such that both the strands/yarns 1060 and the strands/yarns 1062 curve around the weft strands/yarns 1064. FIG. 19D illustrates another exemplary leno weave lacing pattern 502 produced when multiple warp beams are used and the leno strands/yarns 1060 are less tensioned than the standard strands/yarns 1062 such that the standard strands/yarns 1062 remain relatively straight in the weave, and perpendicular to the weft strands/yarns 1064, while the leno strands/yarns 1060 curve around the standard strands/yarns 1062.

FIG. 19E illustrates a leno weave interlacing pattern 504, which is similar to pattern 500 FIG. 19C, but in which alternate leno strands/yarns 1060 are point-drafted (for example, a technique in which the leno strands/yarns are drawn through heddles) such that adjacent leno strands/yarns 1060 have opposing lacing directions. FIG. 19F illustrates a leno weave interlacing pattern 506 similar to pattern 502 FIG. 19D, but in which the leno strands/yarns 1060 are point-drafted such that adjacent leno strands/yarns have opposite lacing directions.

FIG. 19G is a cross-sectional view of a leno weave structure taken through the weft strands/yarns 1064, which may be either of the leno weave patterns 500 or 504.

FIG. 19J illustrates a representative section of the leno weave pattern 500 of FIG. 19C as viewed from the reverse side of the fabric.

Further details regarding fabric skirts that include leno weaves which may be utilized with the skirts described herein are disclosed in U.S. Pat. No. 11,013,600 and U.S. Patent Publication No. 2019/0374337, which are each incorporated by reference herein.

### Features Related to Increased Stretchability/Flexibility and Reduced Crimp Profile

As discussed above, it is desirable that encapsulated skirts (such as for example, the encapsulated inner skirt 16 and the encapsulated outer skirt 202) exhibit sufficient elongation or stretchability (flexibility) in the axial direction so as not to inhibit elongation of the frame during crimping of the prosthetic valve. Further, it is desirable to have a reduced overall crimp profile of a prosthetic valve to enable improved transcatheter delivery thereof. However, encapsulation of the fabric layer (that is, the reinforcing layer) of the skirt can decrease flexibility and/or stretchability of the skirt, as well as increase its thickness thereby contributing to an overall increase in the crimp profile of the prosthetic valve when attached thereto. Moreover, decreased flexibility of encapsulated skirts may negatively impact their functionality after implantation of the prosthetic valve. For example, as discussed above, the inner and/or outer skirts may function as sealing members and reduce or avoid perivalvular leakage. Thus, overly stiff encapsulated skirt material may reduce the ability of the skirt (for example, outer skirt) to conform to anatomy of a patient and form a sufficient seal.

Accordingly, the encapsulated skirts described herein can be formed and include features to address one or more of the foregoing issues. For example, the encapsulated skirts described herein can have an increased flexibility or stretchability and/or a decreased thickness relative to conventional encapsulated skirts and improve elongation of the skirts when a prosthetic valve is in a crimped state. Further, in examples, the encapsulated skirts described herein can have a reduced volume when compressed that contributes to a reduced crimp profile of the prosthetic valve. Yet further, the increased flexibility of the encapsulated skirts described herein can improve formation of non-developable surfaces or complex curved shapes after implantation and radial expansion of the prosthetic valve, thereby enabling the skirt to for example, conform to native anatomy and form a sufficient seal between the prosthetic valve and the native anatomy.

For example, each encapsulating layer (for example, a TPU or other thermoplastic encapsulating layer) can be provided with or made to have a reduced thickness. Further, in examples, the yarns or filaments can include a leno weave pattern that enables the yarns or filaments to have greater degree of spacing (that is, a greater distance therebetween).

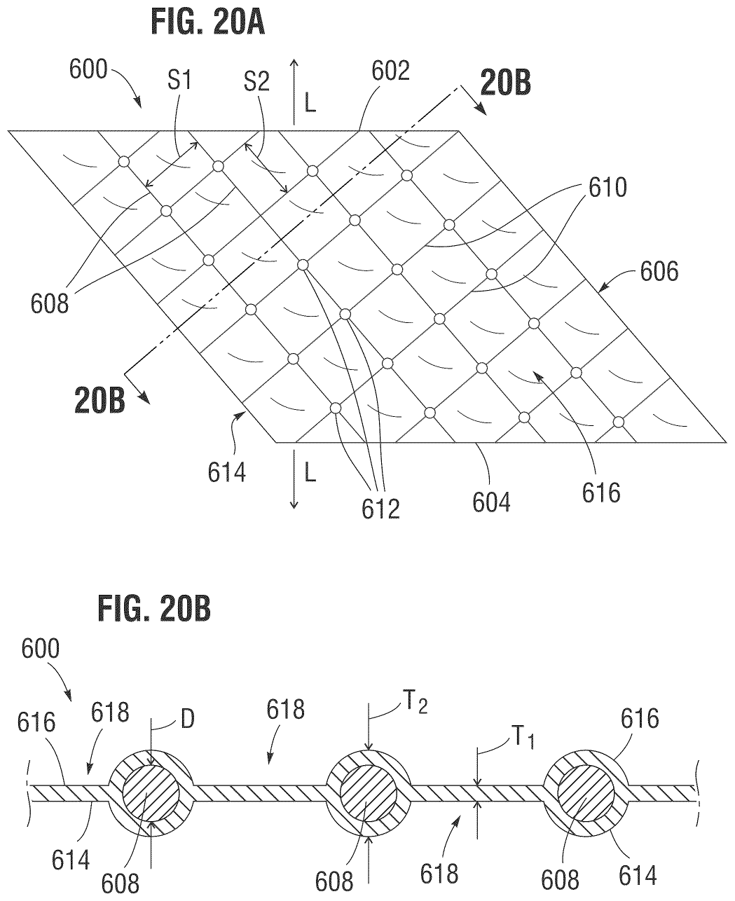

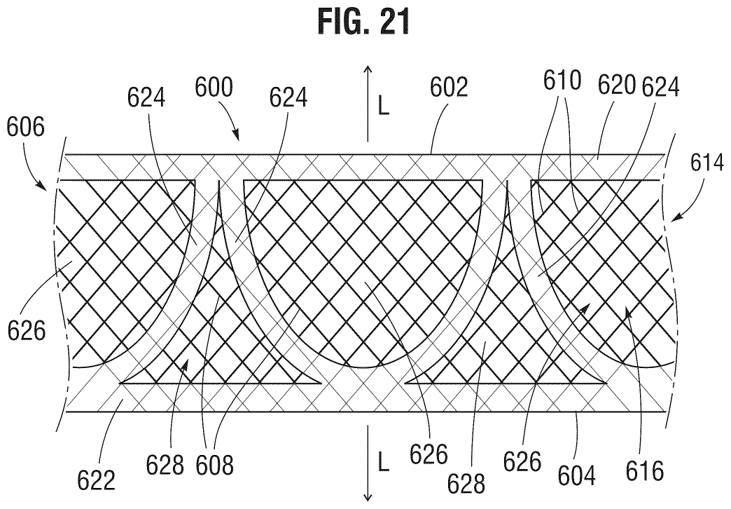

FIG. 20A illustrates an exemplary section of an encapsulated skirt 600 (which may be for example, an outer skirt or an inner skirt). In examples, a top edge 602 of the skirt 600 is oriented towards an outflow end of a prosthetic valve (such as for example, the prosthetic valve 10 or 200) and a lower edge 604 is oriented towards an inflow end of the prosthetic valve. The exemplary skirt 600 comprises a reinforcing layer 606 including a first plurality of yarns 608 (which can be filaments, yarns, or threads) interwoven with and perpendicular to a second plurality of yarns 610 (which can be filaments, yarns, or threads of a same or different type, such as a less elastic type, relative to yarns 608). A plurality of junctions 612 are formed where the yarns 608, 610 intersect and are woven together. Similar to the example described above with reference to FIGS. 16A and 16B, the yarns 608, 610 can be disposed at non-perpendicular and non-parallel angles relative to the longitudinal axis L of the prosthetic valve (for example, the yarns are disposed at an approximately 45-degree angle orientation relative to a longitudinal axis L) when the skirt 600 is in a relaxed state (corresponding to the radially expanded state of the prosthetic valve), and can be movable to an axially elongated position where the yarns are closer to parallel an orientation with the longitudinal axis L when the skirt 600 is in an axially stretched or elongated state (corresponding to the radially compressed state of the prosthetic valve, such as after crimping).

As discussed above, in plain weave patterned fabrics or reinforcing layers typically have a minimum weave density of about 160 ppi in order to maintain structure integrity of fabric. In order to increase flexibility and decrease thickness of a skirt 600 including the illustrated section, the yarns 608, 610 in the reinforcing layer 606 can comprise a leno weave pattern, such as one or more of the leno weave patterns discussed above with reference to FIGS. 19A-19J. For example, each yarn 608 can represent a yarn 1064 (for example, a weft yarn) and each yarn 610 can represent a pair of yarns 1062, 1064 (for example, a pair of warp yarns woven around a weft yarn 608 to form junctions 612). Accordingly, in examples, the reinforcing layer 606 can have a weave density of about 10 to about 60 ppi. Further, in examples, a spacing S1 and S2 between adjacent yarns 608 and adjacent yarns 610, respectively, can be at least 0.5 mm, at least 0.6 mm, at least 0.7 mm, at least 0.8 mm, at least 0.9 mm, or at least 1.0 mm. In certain examples, the spacing S1, S2 can be in the range of about 0.5 mm to about 2.0 mm, more specifically in the range of about 1.0 mm to about 2.0 mm. Moreover, the leno weave pattern can create fixed positioning of the yarns 608, 610 at the junctions 612 in order to retain the spacing and positioning of the threads in the weaver pattern when transitioning between axially stretched or elongated state and the relaxed state.

The decreased weave density can increase flexibility or stretchability of the skirt 600 (relative to conventional skirt), and can additionally decrease (relative to conventional skirt) an overall bulk or volume of the skirt, thereby reducing an overall crimp profile of the prosthetic valve to which it is attached.

Additionally or alternatively, the skirt 600 can be provided with or formed so as to have relatively thin encapsulating layers 614, 616 (for example, TPU or other thermoplastic encapsulating layers) disposed on interior and exterior surfaces of the reinforcing layer 606. In examples, one or more of each of the encapsulating layers 614, 616 can have a thickness in the range of about 5 μm to about 15 μm, which may be similar to a thickness of the reinforcing layer yarns 608, 610. For example, as discussed above, a thickness of the yarns can be in the range of about 8 μm to about 20 μm, with 10 μm being a specific example. In alternate examples, the yarns 608, 610 can be thicker (for example, up to 30 μm) or thinner (for example, as low as 5 μm) than the encapsulating layers 614, 616. At the junctions 612, the skirt 600 may have an increased thickness relative to other regions of the skirt, as the yarns 608, 610 overlap or are interwoven via the leno weave pattern at each of the junctions. For example, the junctions 612 may have a thickness of in the range of about 30 μm to about 150 μm, depending on for example, the diameters of the yarns 608, 610, the thickness of each of the encapsulating layers 614, 616, and/or the specific weave pattern of the reinforcing layer 606.

FIG. 20B shows an exemplary cross section of skirt 600 taken along the line B-B shown in FIG. 20A. As can be seen therein, areas 618 between adjacent yarns 608 of the skirt 600 (formed by the bonded encapsulating layers 614, 616) have a thickness T1 (for example, in the range of about 10 μm to about 30 μm, and with 20 μm being a specific example). Each of the yarns 608 has a diameter D (for example, in the range of about 8 μm to about 20 μm), and the thickness of the skirt 600 at the yarns 608 is increased via having the encapsulating layers 614, 616 disposed thereon. Accordingly, at each of the yarns 608 the skirt 600 has a total thickness T2 (for example, in the range of about 18 μm to about 46 μm). The total thickness T2 can vary depending on the thickness of the layers 614, 616 and the thickness of the yarns 608, 610.

The relatively thinner areas 618 between the yarns can further increase flexibility or stretchability of the skirt 600 (relative to conventional skirt), and can additionally decrease (relative to conventional skirt) an overall bulk or volume of the skirt, thereby reducing an overall crimp profile of the prosthetic valve to which it is attached.

Additionally or alternatively, flexibility or stretchability of the skirt 600 can be increased by reducing a degree or a total area of bonding between the yarns 608, 610 of the reinforcing layer 606 and the encapsulating layers 614, 616. In examples, the encapsulating layers 614, 616 can be attached or bonded at selected regions or strips to allow the reinforcing layer 606 to move freely between the encapsulating layers 614, 616 at areas that are unattached or unbonded. In other words, one or more pockets may be created between the encapsulating layers 614, 616 where the reinforcing layer 606 is unbonded and can move without restriction between axially elongated and relaxed states.

One specific example is illustrated in FIG. 21, wherein the skirt 600 includes a first encapsulated region 620 that extends along the top edge 602 of the skirt and a second encapsulated region 622 that extends along the bottom edge 604 of the skirt. At each of the first and second encapsulated regions 620, 622, the encapsulating layers 614, 616 are bonded to each other and to the surfaces of sections of the yarns 608, 610 that lay within regions 620, 622. The encapsulating at the bottom and top edges 602, 604 of the skirt 600 can seal the reinforcing layer 606 within the encapsulating layers 614, 616, can prevent or limit lateral and/or axial sliding or displacement of the reinforcing layer between the encapsulating layer 614, 616, and can prevent unraveling of the yarns 608, 610 at the opposing edges of the skirt.

The skirt 600 can optionally include one or more additional encapsulated regions. For example, the skirt 600 can include a third encapsulated region comprising one or more U-shaped regions 624 that extend along a region of the skirt corresponding to a scallop line of a valvular structure (such as for example, the U-shaped inlet or cusp edge 52 the valvular structure 14 shown in FIG. 2). Each U-shaped region 624 has a size and shape that corresponds to a cusp edge portion of a leaflet 40. Thus, there can be a U-shaped region 624 for each leaflet 40 (for example, three leaflets 40 and three U-shaped regions). The cusp edge portions of the leaflets 40 optionally can be secured (for example, by sutures) to the skirt along the U-shaped regions 624. For example, if the skirt 600 is an inner skirt of a prosthetic valve, the cusp edge portions of the leaflets typically are sutured to the skirt along the U-shaped regions 624.

Accordingly, pockets 626, 628 can be formed between the layers 614, 616 in the regions or areas where the layers 614, 616 are not bonded to each other. Each pocket 626 can be bounded by a U-shaped region 624 and a section of the first encapsulated region 620. Each pocket 628 can be bounded by two adjacent U-shaped regions 624 and a section of the second encapsulated region 622. The encapsulated regions 620, 622, and 624 can be referred to as bonded regions of the skirt assembly. The pockets 626, 628 can be referred to as un-bonded regions of the skirt assembly. The reinforcing layer 606 can be retained within the pockets 626, 628, and, by being unattached to the encapsulating layers 614, 616, move freely between relaxed and axially elongated states within the pockets during transition of the prosthetic valve between radially expanded and crimped states. Additionally, the bonded regions are relatively less stretchable than the unbonded regions. Advantageously, if the cusp edge portions of the leaflets are secured (for example, sutured) to the skirt along the U-shaped-regions 624, they can prevent or minimize stretching of the leaflets 40 in the axial direction when the prosthetic valve is radially compressed.

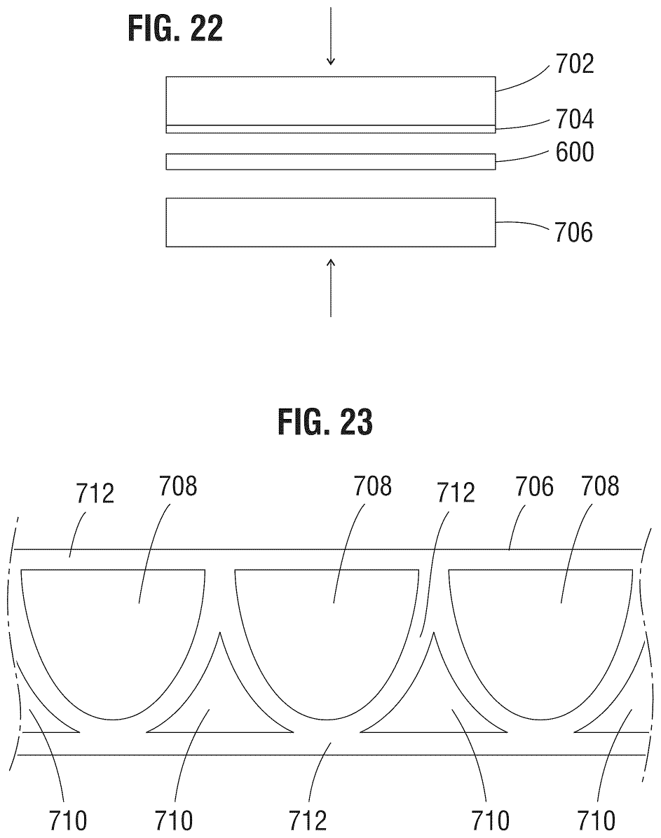

Referring to FIG. 22, methods for forming the skirt 600 can include utilizing a pressing plate assembly comprising a first flat plate 702, which can have a slightly compressible surface layer 704 disposed over an inner surface the first plate (for example, a silicon layer disposed over surface of a flat metal plate), and a second plate 706. The three layers of the skirt (that is, the first encapsulating layer 614, the second encapsulating layer 616, and the reinforcing layer 606 disposed therebetween) can be placed between the first and second plates. All tree layers are compressed between the first and second plates 702, 706 under a specified pressure and/or elevated temperatures to bond the encapsulating layers to each other and the reinforcing layer, thereby encapsulating each of the filaments or yarns of the skirt. The second plate optionally can include a slightly compressible layer (for example, a silicone layer) that contacts an adjacent surface of the skirt assembly during the encapsulation process. The process of applying pressure and/or heat via the plates can make the material forming the layers 614, 616 flowable and forms the thinner areas 618 between the yarns 608, 610 with a desired thickness.

Referring to FIG. 23, for forming a skirt 600 having pockets 626, 628, the second plate 706 includes one or more depressions 708, 710 formed on the inner surface of the plate 706. The depressions 708 correspond in size and shape to the pockets 626 and the depressions 710 correspond in size and shape to the pockets 628. The inner surface of the plate 706 therefore includes protrusions or raised regions 712 surrounding the depressions 708, 710 and corresponding to the bonded regions 620, 622, 624 of the skirt. Accordingly, when the first and second plates 702, 706 are pressed against the three layers of the skirt (that is, the first encapsulating layer, the second encapsulating layer, and the reinforcing layer disposed therebetween), the raised regions 712 of the second plate press against the skirt layers and cause bonding at the specified regions (for example, regions 620, 622, 624) to bond the layers 614, 616 to each other along those regions, while the depressed regions 708, 710 avoid the application of pressure and/or heat to the remainder of the skirt by the plates 702, 706, thereby forming the pockets 620, 622, 624 in which the layers 614, 616 are un-bonded yet retain reinforcing layer 606.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above described examples of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic valve comprising: an annular frame that is radially expandable from a radially compressed state to a radially expanded state, the annular frame having an inflow end, an outflow end, and a longitudinal axis extending from the inflow end to the outflow end; a plurality of leaflets configured to regulate a flow of blood from the inflow end to the outflow end of the annular frame; and a skirt assembly comprising a reinforcing layer sandwiched between a first encapsulating layer and a second encapsulating layer, wherein the reinforcing layer comprises a textile comprising a first set of yarns and a second set of yarns interwoven with the first set of yarns; wherein the skirt assembly has a first thickness at a plurality of relatively thinner areas between adjacent yarns and a second thickness at the yarns, wherein the first thickness is less than the second thickness.

Example 2. The prosthetic heart valve of any example herein, particularly example 1, wherein yarns in each of the first set of yarns and the second set of yarns are non-perpendicular and non-parallel to the longitudinal axis when the annular frame is in the radially expanded state.

Example 3. The prosthetic heart valve of any example herein, particularly any of examples 1 or 2, wherein the skirt assembly is configured such that, when the annular frame is in the radially expanded state, yarns in each of the first set of yarns and the second set of yarns are oriented at angles in a range of about 20 degrees to about 70 degrees relative to the longitudinal axis of the annular frame.

Example 4. The prosthetic heart valve of any example herein, particularly any of examples 1-3, wherein the skirt assembly is configured such that, when the annular frame is in the radially expanded state, yarns in each of the first set of yarns and the second set of yarns are oriented at 45-degree angles relative to the longitudinal axis of the frame.

Example 5. The prosthetic heart valve of any example herein, particularly any of examples 1-4, wherein each of the first encapsulating layer and the second encapsulating layer is made of an elastomer.

Example 6. The prosthetic heart valve of any example herein, particularly any of examples 1-5, wherein the each of the first and second encapsulating layer and has a thickness in a range of about 5 μm to about 15 μm.

Example 7. The prosthetic heart valve of any example herein, particularly any of examples 1-6, wherein yarns of the first set are interwoven with the yarns of the second set in a leno weave pattern.

Example 8. The prosthetic heart valve of any example herein, particularly any of examples claims 1-7, wherein the textile has a weave density of less than 150 ppi.

Example 9. The prosthetic heart valve of any example herein, particularly example 8, wherein the textile layer has a weave density of 60 ppi or less.

Example 10. The prosthetic heart valve of any example herein, particularly example 9, wherein the textile layer has a weave density from about 10 to about 60 ppi.

Example 11. The prosthetic heart valve of any example herein, particularly any of examples 1-10, wherein spacing between adjacent yarns in the first set of yarns is at least 0.5 mm.

Example 12. The prosthetic heart valve of any example herein, particularly example 11, wherein the spacing between adjacent yarns in the first set of yarns is in a range of about 0.5 mm to about 2.0 mm.

Example 13. The prosthetic heart valve of any example herein, particularly any of examples 1-12, wherein spacing between adjacent yarns in the second set of yarns is at least 0.5 mm.

Example 14. The prosthetic heart valve of any example herein, particularly example 13, wherein spacing between adjacent yarns in the second set of yarns is in a range of about 0.5 mm to about 2.0 mm.

Example 15. The prosthetic heart valve of any example herein, particularly any of examples 1-14, wherein yarns in the first set of yarns have a thickness or diameter in a range of about 8 μm to about 16 μm.

Example 16. The prosthetic heart valve of any example herein, particularly any of examples 1-15, wherein yarns in the second set of yarns have a thickness or diameter in a range of about 8 μm to about 16 μm.

Example 17. The prosthetic heart valve of any example herein, particularly any of examples 1-16, wherein the first encapsulating layer is bonded to the second encapsulating layer at the plurality of relatively thinner areas.

Example 18. The prosthetic heart valve of any example herein, particularly any of examples 1-17, wherein the first thickness is in a range of about 10 μm to about 30 μm.

Example 19. The prosthetic heart valve of any example herein, particularly any of examples 1-18, wherein the skirt assembly is configured such that the first encapsulating layer and the second encapsulating layer are bonded together along one or more bonded regions, and one or more pockets are formed in areas between the one or more bonded regions, wherein the first and second encapsulating layers are unbonded to each other within the pockets.

Example 20. The prosthetic heart valve of any example herein, particularly example 19, wherein the reinforcing layer is secured to the first and second encapsulating layers within the one or more bonded regions, and sections of the reinforcing layer within the pockets are disposed between the first encapsulating layer and the second encapsulating layer but unattached thereto.

Example 21. The prosthetic heart valve of any example herein, particularly examples 19 and 20, wherein the one or more pockets are configured such that the sections of the reinforcing layer within the pockets can move relative to the first and second encapsulating layers within a respective pocket.

Example 22. The prosthetic heart valve of any example herein, particularly any of examples 19-21, wherein the one or more bonded regions comprise a first bonded region along a first circumferential edge of the skirt assembly and a second bonded region along a second circumferential edge of the skirt assembly, the first edge opposing the second edge.

Example 23. The prosthetic heart valve of any example herein, particularly any of examples 19-22, wherein the one or more bonded regions comprise one or more U-shaped bonded regions corresponding to at least a portion of a scallop line of the plurality of leaflets.

Example 24. The prosthetic heart valve of any example herein, particularly any of examples 1-23, wherein the skirt assembly is configured such that, when the annular frame is in the radially compressed state, the skirt assembly is elongated axially up to at least 40% of its initial length when the annular frame is in the radially expanded state.

Example 25. A prosthetic valve comprising: an annular frame that is radially expandable from a radially compressed state to a radially expanded state, the annular frame having an inflow end, an outflow end, and a longitudinal axis extending from the inflow end to the outflow end; a plurality of leaflets configured to regulate a flow of blood from the inflow end to the outflow end of the annular frame; and a skirt assembly comprising a reinforcing layer sandwiched between a first encapsulating layer and a second encapsulating layer, wherein the reinforcing layer comprises a textile comprising a first set of yarns and a second set of yarns interwoven in a leno weave pattern with the first set of yarns, wherein the first set of yarns and the second set of yarns are each non-perpendicular and non-parallel to the longitudinal axis when the annular frame is in the radially expanded state; wherein each of the first encapsulating layer and the second encapsulating layer is made of an elastomer and has a thickness in a range of about 5 μm to about 15 μm.

Example 26. The prosthetic heart valve of any example herein, particularly example 25, wherein each of the first encapsulating layer and the second encapsulating layer has a thickness in a range of about 5 μm to about 10 μm.

Example 27. The prosthetic heart valve of any example herein, particularly examples 25 and 26, wherein the textile has a weave density of less than 150 ppi.

Example 28. The prosthetic heart valve of any example herein, particularly example 27, wherein the textile has a weave density of 60 ppi or less.

Example 29. The prosthetic heart valve of any example herein, particularly example 28, wherein the textile has a weave density from about 10 to about 60 ppi.

Example 30. The prosthetic heart valve of any example herein, particularly any of examples 25-29, wherein spacing between adjacent yarns in the first set of yarns is at least 0.5 mm.

Example 31. The prosthetic heart valve of any example herein, particularly example 30, wherein the spacing between adjacent yarns in the first set of yarns is in a range of about 1.0 mm to about 2.0 mm.

Example 32. The prosthetic heart valve of any example herein, particularly any of examples 25-31, wherein spacing between adjacent yarns in the second set of yarns is at least about 0.5 mm.

Example 33. The prosthetic heart valve of any example herein, particularly example 32, wherein the spacing between adjacent yarns in the second set of yarns is in a range of about 1.0 mm to about 2.0 mm.

Example 34. The prosthetic heart valve of any example herein, particularly any of examples 25-33, wherein each yarn in the first set of yarns has a thickness or diameter in a range of about 8 μm to about 16 μm.

Example 35. The prosthetic heart valve of any example herein, particularly any of examples 25-34, wherein each yarn in the second set of yarns has a thickness or diameter in a range of about 8 μm to about 16 μm.

Example 36. The prosthetic heart valve of any example herein, particularly any of examples 25-35, wherein the skirt assembly is configured such that, when the annular frame is in the radially expanded state, yarns in each of the first set of yarns and the second set of yarns are oriented at angles in a range of about 20 degrees to about 70 degrees relative to the longitudinal axis of the annular frame.

Example 37. The prosthetic heart valve of any example herein, particularly example 36, wherein the skirt assembly is configured such that, when the annular frame is in the radially expanded state, yarns in each of the first set of yarns and the second set of yarns are oriented at 45-degree angles relative to the longitudinal axis of the frame.

Example 38. The prosthetic heart valve of any example herein, particularly any of examples 25-37, wherein the skirt assembly further comprises a plurality of relatively thinner areas between adjacent yarns where the first encapsulating layer is bonded directly to the second encapsulating layer.

Example 39. The prosthetic heart valve of any example herein, particularly example 38, wherein each of the plurality of thinner areas have a thickness in a range of about 10 μm to about 30 μm.

Example 40. The prosthetic heart valve of any example herein, particularly example 39, wherein each of the plurality of thinner areas have a thickness in a range of about 10 μm to about 20 μm.

Example 41. The prosthetic heart valve of any example herein, particularly any of examples 25-37, wherein the skirt assembly is configured such that the first encapsulating layer and the second encapsulating layer are bonded together along one or more bonded regions, and one or more pockets are formed in areas between the one or more bonded regions, wherein the first and second encapsulating layers are unbonded to each other within the pockets.

Example 42. The prosthetic heart valve of any example herein, particularly example 41, wherein the reinforcing layer is secured to the first and second encapsulating layers within the one or more bonded regions, and sections of the reinforcing layer within the pockets are disposed between the first encapsulating layer and the second encapsulating layer but unattached thereto.

Example 43. The prosthetic heart valve of any example herein, particularly examples 41 and 42, wherein the one or more pockets are configured such that the sections of the reinforcing layer within the pockets can move relative to the first and second encapsulating layers within a respective pocket.

Example 44. The prosthetic heart valve of any example herein, particularly any of examples 41-43, wherein the one or more bonded regions comprise a first bonded region along a first circumferential edge of the skirt assembly and a second bonded region along a second circumferential edge of the skirt assembly, the first edge opposing the second edge.

Example 45. The prosthetic heart valve of any example herein, particularly any of examples 41-44, wherein the one or more bonded regions comprise one or more U-shaped bonded regions corresponding to at least a portion of a scallop line of the plurality of leaflets.

Example 46. The prosthetic heart valve of any example herein, particularly any of examples 41-45, wherein skirt assembly comprises an outer skirt mounted on an exterior surface of the annular frame, the outer skirt configured to form a snug fit with the annular frame such that the outer skirt lies against the outer surface when the annular frame is in the radially expanded state.

Example 47. The prosthetic heart valve of any example herein, particularly any of examples 25-46, wherein the skirt assembly is configured such that, when the annular frame is in the radially compressed state, the skirt assembly is elongated axially up to at least 40% of its initial length when the annular frame is in the radially expanded state.

Example 48. A prosthetic valve comprising: an annular frame that is configured to be radially expanded from a radially compressed state to a radially expanded state, the annular frame having an inflow end and an outflow end; a plurality of leaflets configured to regulate a flow of blood therethrough; and a skirt assembly comprising a reinforcing layer sandwiched between a first encapsulating layer and a second encapsulating layer; wherein the skirt assembly is configured such that the first encapsulating layer and the second encapsulating layer are fused along one or more bonded regions, and one or more pockets are formed in areas between the one or more bonded regions, thereby enabling sections of the reinforcing layer within the one or more pockets to be unattached to the first encapsulating layer and the second encapsulating layer.

Example 49. The prosthetic heart valve of any example herein, particularly example 48, wherein skirt assembly comprises an outer skirt mounted on an exterior surface of the annular frame, the outer skirt configured to form a snug fit with the annular frame such that the outer skirt lies against the outer surface when the annular frame is in the radially expanded state.

Example 50. The prosthetic heart valve of any example herein, particularly examples 48 and 49, wherein the reinforcing layer comprises a textile comprising a first set of yarns and a second set of yarns interwoven in a weave pattern with the first set of yarns, and wherein the first set of yarns and the second set of yarns are each non-perpendicular and non-parallel to a longitudinal axis of the frame.

Example 51. The prosthetic heart valve of any example herein, particularly example 50, wherein the weave pattern is a leno weave pattern.

Example 52. The prosthetic heart valve of any example herein, particularly examples 50 and 51, wherein the skirt assembly is configured such that, when the annular frame is in the radially expanded state, yarns in each of the first set of yarns and the second set of yarns are oriented at angles in a range of 20 to 70 degrees relative to the longitudinal axis of the annular frame.

Example 53. The prosthetic heart valve of any example herein, particularly example 52, wherein the skirt assembly is configured such that, when the annular frame is in the radially expanded state, the yarns in each of the first set of yarns and the second set of yarns are oriented at about 45-degree angles relative to the longitudinal axis of the annular frame.

Example 54. The prosthetic heart valve of any example herein, particularly any of examples 50-53, wherein the textile has a weave density of less than 150 ppi.

Example 55. The prosthetic heart valve of any example herein, particularly example 54, wherein the textile has a weave density of 60 ppi or less.

Example 56. The prosthetic heart valve of any example herein, particularly example 55, wherein the textile has a weave density in a range of about 10 to about 60 ppi.

Example 57. The prosthetic heart valve of any example herein, particularly any of examples 50-56, wherein spacing between adjacent yarns in the first set of yarns is at least about 0.5 mm.

Example 58. The prosthetic heart valve of any example herein, particularly example 57, wherein the spacing between adjacent yarns in the first set of yarns is in a range of about 1.0 mm to about 2.0 mm.

Example 59. The prosthetic heart valve of any example herein, particularly any of examples 50-58, wherein spacing between adjacent yarns in the second set of yarns is at least about 0.5 mm.

Example 60. The prosthetic heart valve of any example herein, particularly example 59, wherein the spacing between adjacent yarns in the second set of yarns is in a range of about 1.0 mm to about 2.0 mm.

Example 61. The prosthetic heart valve of any example herein, particularly any of examples 48-60, wherein each of the first encapsulating layer and the second encapsulating layer has a thickness in a range of about 5 μm to about 15 μm.

Example 62. The prosthetic heart valve of any example herein, particularly example 61, wherein each of the first encapsulating layer and the second encapsulating layer has a thickness in a range of about 5 μm to about 10 μm.

Example 63. The prosthetic heart valve of any example herein, particularly any of examples 48-62, wherein the reinforcing layer is attached to the first and second encapsulating layers within the one or more bonded regions.

Example 64. The prosthetic heart valve of any example herein, particularly any of examples 48-63, wherein the one or more pockets are configured such that the sections of the reinforcing layer within the pockets can move relative to the first and second encapsulating layers.

Example 65. The prosthetic heart valve of any example herein, particularly any of examples 48-64, wherein the one or more bonded regions comprise a first bonded strip along a first circumferential edge of the skirt assembly and a second bonded strip along a second circumferential edge of the skirt assembly, the first edge opposing the second edge.

Example 66. The prosthetic heart valve of any example herein, particularly any of examples 48-65, wherein the one or more bonded regions comprise one or more U-shaped bonded strips corresponding to at least a portion of a scallop line of the plurality of leaflets.

Example 67. A skirt assembly for a prosthetic valve, the prosthetic valve comprising an annular frame that is configured to be radially expanded from a radially compressed state to a radially expanded state, the annular frame having an inflow end, an outflow end, and a longitudinal axis extending from the inflow end to the outflow end, and a plurality of leaflets configured to regulate a flow of blood from the inflow end to the outflow end of the annular frame, the skirt assembly configured to be attached to a surface of the annular frame and to be moveable between a relaxed state and an axially elongated state, the skirt assembly comprising: a first encapsulating layer and a second encapsulating layer, each of the first encapsulating layer and the second encapsulating layer comprising an elastomeric material; and a reinforcing layer disposed between the first encapsulating layer and the second encapsulating layer, wherein the reinforcing layer comprises a textile comprising a first set of yarns and a second set of yarns interwoven with the first set of yarns, wherein the first set of yarns and the second set of yarns are each non-perpendicular and non-parallel to a top edge and a bottom edge of the skirt assembly when the skirt assembly is in the relaxed state; wherein the skirt assembly is configured such that the first encapsulating layer and the second encapsulating layer are fused along one or more bonded regions, thereby forming one more pocket regions between the encapsulating first layer and the second encapsulating layer, wherein the first and second encapsulating layers are not fused to each other within the pockets regions.

Example 68. The skirt assembly of any example herein, particularly example 67, wherein each of the first encapsulating layer and the second encapsulating layer have a thickness in a range of about 5 μm to about 15 μm.

Example 69. The skirt assembly of any example herein, particularly examples 67 and 68, wherein the first set of yarns and the second set of yarns are interwoven in a leno weave pattern.

Example 70. The skirt assembly of any example herein, particularly any of examples 67-69, wherein spacing between adjacent yarns in the first set of yarns is at least 0.5 mm.

Example 71. The skirt assembly of any example herein, particularly example 70, wherein spacing between adjacent yarns in the first set of yarns is in a range of about 0.5 mm to about 2.0 mm.

Example 72. The skirt assembly of any example herein, particularly any of examples 67-71, wherein spacing between adjacent yarns in the second set of yarns is in a range of about 0.5 mm to about 2.0 mm.

Example 73. The skirt assembly of any example herein, particularly any of examples 67-72, wherein the skirt assembly is configured such that, when the skirt assembly is in the relaxed state, yarns in each of the first set of yarns and the second set of yarns are oriented at about 45-degree angles relative to the top edge and the bottom edge of the skirt assembly.

Example 74. The skirt assembly of any example herein, particularly any of examples 67-73, wherein the one or more pocket regions are configured such that sections of the reinforcing layer outside of the one or more one or more bonded regions are disposed between the first encapsulating layer and the second encapsulating layer but unattached thereto.

Example 75. The skirt assembly of any example herein, particularly any of examples 67-74, wherein the one or more bonded regions comprise a first bonded region along the top edge of the skirt assembly and a second bonded region along the bottom edge of the skirt assembly.

Example 76. The skirt assembly of any example herein, particularly example 75, wherein the reinforcing layer is sealed within the first encapsulating layer and the second encapsulating layer via the first bonded region and the second bonded region.

Example 77. The skirt assembly of any example herein, particularly examples 75 and 76, wherein the first bonded region and the second bonded region limit lateral and axial movement of the reinforcing layer within the first encapsulating layer and the second encapsulating layer.

Example 78. The skirt assembly of any example herein, particularly any of examples 67-77, wherein the one or more bonded regions comprise one or more U-shaped bonded regions corresponding to at least a portion of a scallop line of the plurality of leaflets.

Example 79. The skirt assembly of any example herein, particularly any of examples 67-78, wherein the skirt assembly is an outer skirt configured to be attached on an outer surface of the annular frame.

Example 80. The skirt assembly of any example herein, particularly any of examples 67-79, wherein the skirt assembly is configured such that, when the annular frame is in the radially compressed state, the skirt assembly is elongated axially up to at least 40% of its initial length when the annular frame is in the radially expanded state.

Example 81. A method of assembling a prosthetic valve comprising: mounting a plurality of leaflets to an annular frame that is radially expandable from a radially compressed state to a radially expanded state, wherein the frame has an inflow end, an outflow end, and a longitudinal axis extending from the inflow end to the outflow end, wherein the plurality of leaflets are configured to regulate a flow of blood from the inflow end to the outflow end of the frame; forming a skirt assembly comprising a woven reinforcing layer sandwiched between a first encapsulation layer and a second encapsulation layer, the forming comprising fusing the first encapsulation layer and the second encapsulation layer along one or more fused regions, the one or more fused regions comprising a first region along a top edge of the skirt assembly and a second region at along a bottom edge of the skirt assembly; and mounting the skirt assembly to the annular frame.

Example 82. The method of any example disclosed herein, particularly example 81, wherein the skirt assembly comprises an outer skirt, and the mounting the skirt assembly to the annular frame comprises mounting the outer skirt on an outer surface of the annular frame.

Example 83. The method of any example disclosed herein, particularly example 81, wherein the skirt assembly comprises an inner skirt, and the mounting the skirt assembly to the annular frame comprises mounting the inner skirt on an inner surface of the annular frame.

Example 84. The method of any example disclosed herein, particularly any one of examples 81-83, wherein the reinforcing layer comprises a textile comprising a first set of yarns and a second set of yarns interwoven in a leno weave pattern with the first set of yarns, wherein the mounting the skirt assembly to the annular frame comprises mounting the skirt assembly such that the first set of yarns and the second set of yarns are each non-perpendicular and non-parallel to the longitudinal axis when the annular frame is in the radially expanded state.

Example 85. The method of any example disclosed herein, particularly example 84, wherein spacing between adjacent yarns in the first set of yarns is in a range of about 0.5 mm to about 2.0 mm.

Example 86. The method of any example disclosed herein, particularly examples 84 and 85, wherein spacing between adjacent yarns in the second set of yarns is in a range of about 0.5 mm to about 2.0 mm.

Example 87. The method of any example disclosed herein, particularly any of examples 84-86, wherein the forming the skirt assembly comprises positioning the reinforcing layer such that the yarns in each of the first set of yarns and the second set of yarns are oriented at about 45-degree angles relative to top and bottom edges of the skirt assembly.

Example 88. The method of any example disclosed herein, particularly any of examples 81-87, wherein each of the first encapsulating layer and the second encapsulating layer have a thickness in a range of about 5 μm to about 15 μm.

Example 89. The method of any example disclosed herein, particularly any of examples 81-88, wherein the one or more fused regions further comprise one or more U-shaped regions, the one or more U-shaped regions corresponding to at least a portion of a scallop line of the plurality of leaflets.

Example 90. The method of any example disclosed herein, particularly any of examples 81-89, wherein the forming the skirt assembly comprises pressing the skirt assembly between a first plate and a second plate.

Example 91. The method of any example disclosed herein, particularly example 90, further comprising applying heat to the skirt assembly while pressing the skirt assembly between the first and second plates.

Example 92. The method of any example disclosed herein, particularly examples 90 and 91, wherein the first plate comprises a flat plate having a compressible coating.

Example 93. The method of any example disclosed herein, particularly any of examples 99-92, wherein the second plate one or more depressions and one or more raised regions that form the one or more fused regions when the skirt assembly is pressed between the first and second plates.

Example 94. A prosthetic heart valve or skirt assembly for a prosthetic heart valve of any example disclosed herein, particularly any of examples 1-93, wherein the prosthetic heart valve or skirt assembly is sterilized.

The features described herein with regard to any example can be combined with other features described in any one or more of the other examples, unless otherwise stated. For example, any one or more of the features of one outer or inner skirt can be combined with any one or more features of another outer or inner skirt.

In view of the many possible examples to which the principles of the disclosed invention may be applied, it should be recognized that the illustrated examples are only preferred examples of the invention and should not be taken as limiting the scope of the invention. Rather, the scope of the invention is defined by the following claims. We therefore claim as our invention all that comes within the scope and spirit of these claims.

We claim:

1. A prosthetic valve comprising:
an annular frame that is radially expandable from a radially compressed state to a radially expanded state, the annular frame having an inflow end, an outflow end, and a longitudinal axis extending from the inflow end to the outflow end;
a plurality of leaflets configured to regulate a flow of blood from the inflow end to the outflow end of the annular frame; and
a skirt assembly comprising a reinforcing layer sandwiched between a first encapsulating layer and a second encapsulating layer, wherein the reinforcing layer comprises a textile comprising a first set of yarns and a second set of yarns interwoven with the first set of yarns;
wherein the skirt assembly has a first thickness at a plurality of relatively thinner areas between adjacent yarns and a second thickness at the yarns, wherein the first thickness is less than the second thickness.

2. The prosthetic valve of claim 1, wherein yarns in each of the first set of yarns and the second set of yarns are non-perpendicular and non-parallel to the longitudinal axis when the annular frame is in the radially expanded state.

3. The prosthetic valve of claim 1, wherein the skirt assembly is configured such that, when the annular frame is in the radially expanded state, yarns in each of the first set of yarns and the second set of yarns are oriented at 45-degree angles relative to the longitudinal axis of the frame.

4. The prosthetic valve of claim 1, wherein the each of the first and second encapsulating layers is comprised of an elastomer and has a thickness in a range of about 5 μm to about 15 μm.

5. The prosthetic valve of claim 1, wherein yarns in the first set of yarns and in the second set of yarns have a thickness or diameter in a range of about 8 μm to about 16 μm.

6. The prosthetic valve of claim 1, wherein the first encapsulating layer is bonded to the second encapsulating layer at the plurality of relatively thinner areas.

7. The prosthetic valve of claim 1, wherein the first thickness is in a range of about 10 μm to about 30 μm.

8. The prosthetic valve of claim 1, wherein the second thickness is in a range of about 18 μm to about 46 μm.

9. The prosthetic valve of claim 1, wherein the skirt assembly is configured such that the first encapsulating layer and the second encapsulating layer are bonded together along one or more bonded regions, and one or more pockets are formed in areas between the one or more bonded regions, wherein the first and second encapsulating layers are unbonded to each other within the pockets.

10. The prosthetic valve of claim 9, wherein the reinforcing layer is secured to the first and second encapsulating layers within the one or more bonded regions, and sections of the reinforcing layer within the pockets are disposed between the first encapsulating layer and the second encapsulating layer but unattached thereto.

11. The prosthetic valve of claim 1, wherein the skirt assembly is configured such that, when the annular frame is in the radially compressed state, the skirt assembly is elongated axially up to at least 40% of its initial length when the annular frame is in the radially expanded state.

12. A skirt assembly for a prosthetic valve, the prosthetic valve comprising an annular frame that is configured to be radially expanded from a radially compressed state to a radially expanded state, the annular frame having an inflow end, an outflow end, and a longitudinal axis extending from the inflow end to the outflow end, and a plurality of leaflets configured to regulate a flow of blood from the inflow end to the outflow end of the annular frame, the skirt assembly configured to be attached to a surface of the annular frame and to be moveable between a relaxed state and an axially elongated state, the skirt assembly comprising:
a first encapsulating layer and a second encapsulating layer, each of the first encapsulating layer and the second encapsulating layer comprising an elastomeric material; and
a reinforcing layer disposed between the first encapsulating layer and the second encapsulating layer, wherein the reinforcing layer comprises a textile comprising a first set of yarns and a second set of yarns interwoven with the first set of yarns, wherein the first set of yarns and the second set of yarns are each non-perpendicular and non-parallel to a top edge and a bottom edge of the skirt assembly when the skirt assembly is in the relaxed state;
wherein the skirt assembly is configured such that the first encapsulating layer and the second encapsulating layer are fused along one or more bonded regions, thereby forming one more pocket regions between the encapsulating first layer and the second encapsulating layer, wherein the first and second encapsulating layers are not fused to each other within the pocket regions.

13. The skirt assembly of claim 12, wherein the skirt assembly is configured such that, when the skirt assembly is in the relaxed state, yarns in each of the first set of yarns and the second set of yarns are oriented at about 45-degree angles relative to the top edge and the bottom edge of the skirt assembly.

14. The skirt assembly of claim 12, wherein the one or more pocket regions are configured such that sections of the reinforcing layer outside of the one or more one or more bonded regions are disposed between the first encapsulating layer and the second encapsulating layer but unattached thereto.

15. The skirt assembly of claim 12, wherein the one or more bonded regions comprise a first bonded region along the top edge of the skirt assembly and a second bonded region along the bottom edge of the skirt assembly.

16. The skirt assembly of claim 15, wherein the reinforcing layer is sealed within the first encapsulating layer and the second encapsulating layer via the first bonded region and the second bonded region.

17. The skirt assembly of claim 15, wherein the first bonded region and the second bonded region limit lateral and axial movement of the reinforcing layer within the first encapsulating layer and the second encapsulating layer.

18. The skirt assembly of claim 12, wherein the one or more bonded regions comprise one or more U-shaped bonded regions corresponding to at least a portion of a scallop line of the plurality of leaflets.

19. A method of assembling a prosthetic valve comprising:
mounting a plurality of leaflets to an annular frame that is radially expandable from a radially compressed state to a radially expanded state, wherein the frame has an inflow end, an outflow end, and a longitudinal axis extending from the inflow end to the outflow end, wherein the plurality of leaflets are configured to regulate a flow of blood from the inflow end to the outflow end of the frame;
forming a skirt assembly comprising a woven reinforcing layer sandwiched between a first encapsulation layer and a second encapsulation layer, the forming comprising fusing the first encapsulation layer and the second encapsulation layer along one or more fused regions, the one or more fused regions comprising a first region along a top edge of the skirt assembly and a second region at along a bottom edge of the skirt assembly; and
mounting the skirt assembly to the annular frame.

20. The method of claim 19, wherein the forming the skirt assembly comprises positioning the reinforcing layer such that the yarns in each of the first set of yarns and the second set of yarns are oriented at about 45-degree angles relative to top and bottom edges of the skirt assembly.
